(19) United States
(12) Patent Application Publication
(10) Pub. No.: US 2020/0187499 A1
(43) Pub. Date: Jun. 18, 2020

(54) SUBSTITUTED BENZYL-4-AMINOPICOLINIC ESTERS AND PYRIMIDINO-4-CARBOXYLIC ESTERS, METHODS FOR THE PRODUCTION THEREOF, AND USE THEREOF AS HERBICIDES AND PLANT GROWTH REGULATORS

(71) Applicants: Bayer CropScience Aktiengesellschaft, Monheim am Rhein (DE); Bayer Aktiengesellschaft, Leverkusen (DE)

(72) Inventors: Michael Gerhard HOFFMANN, Konstanz (DE); Uwe DOELLER, Rodgau (DE); Chieko UENO, Frankfurt (DE); Hansjoerg DIETRICH, Liederbach am Taunus (DE); Christopher Hugh ROSINGER, Hofheim (DE); Anu Bheemaiah MACHETTIRA, Frankfurt am Main (DE)

(21) Appl. No.: 16/483,672

(22) PCT Filed: Feb. 6, 2018
(86) PCT No.: PCT/EP2018/052911

§ 371 Date: Aug. 5, 2019

(30) Foreign Application Priority Data
Feb. 13, 2017 (EP) 17155824.0

(51) Int. Cl. A01N 43/40; A01N 43/78; C07D 409/10; C07D 405/10; C07D 417/10

(52) U.S. Cl. CPC A01N 43/40; A01N 43/78; C07D 417/10; C07D 405/10; C07D 409/10

(57) ABSTRACT

Substituted benzyl-4-aminopicolinic esters and pyrimidino-4-carboxylic esters, methods for the production thereof, and use thereof as herbicides and plant growth regulators

What is described are benzylcarboxylic acid derivatives of benzoheterocyclylpyridines and benzoheterocyclylpyrimidines of the general formula (I) and their use as herbicides

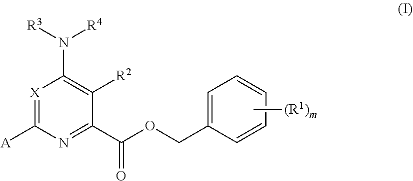

where X represents CH or CF, A represents a benzo-fused heterocycle, R¹ represents H, Hal or organic radicals such as alkyl and R² represents Cl or F.

The invention relates to the technical field of the herbicides, especially that of the herbicides for selective control of weeds and weed grasses in crops of useful plants.

From various publications, substituted picolinic acid derivatives and pyrimidine-4-carboxylic acid derivatives are known to have herbicidal properties: WO 2003/011853 A1 describes polysubstituted 6-phenylpicolinic acid derivatives having herbicidal activity. WO 2009/029735 A1 and WO 2010/125332 A1 describe herbicidal activities of polysubstituted 2-phenyl-4-pyrimidinecarboxylic acid derivatives. Heteroaromatically substituted picolinic and pyrimidinecarboxylic acids having herbicidal properties are disclosed in WO 2009/138712 A2. WO 2013/014165 claims benzoheteroaromatically substituted picolinic and 4-pyrimidinecarboxylic acids as herbicides. WO 2007/080382 A1 and WO 2009/007751 A2 describe heteroaromatically substituted picolinic and pyrimidinecarboxylic acids having pharmacological activities.

However, the compounds described therein frequently have insufficient herbicidal activity and/or insufficient selectivity in crops of useful plants.

We have found substituted benzyl 4-aminopicolinic esters and pyrimidine-4-carboxylic esters which are particularly suitable as herbicides.

The present invention provides benzylpicolinic esters and pyrimidine-4-carboxylic esters of the general formula (I), their N-oxides or their agrochemically acceptable salts,

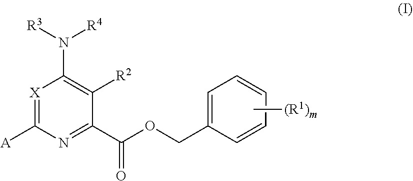

A represents a radical from the group consisting of A1 to A20,

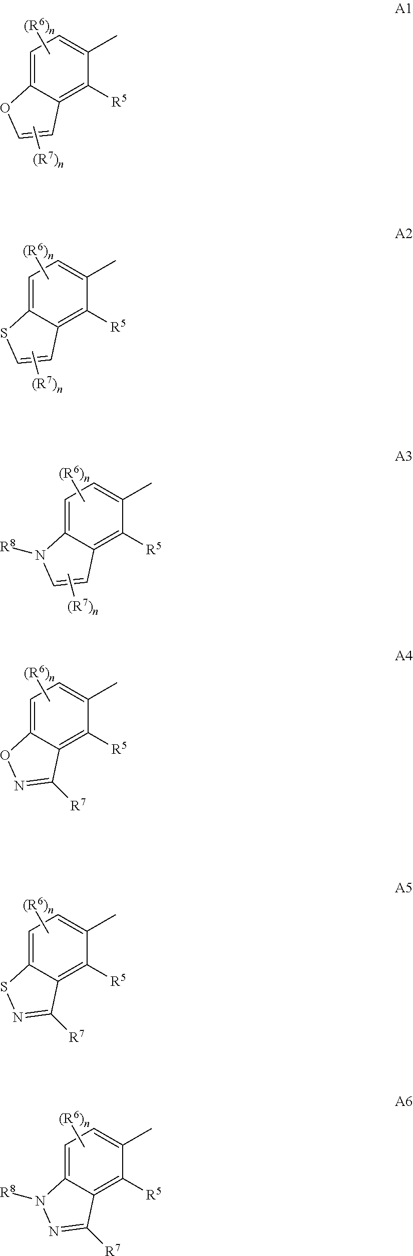

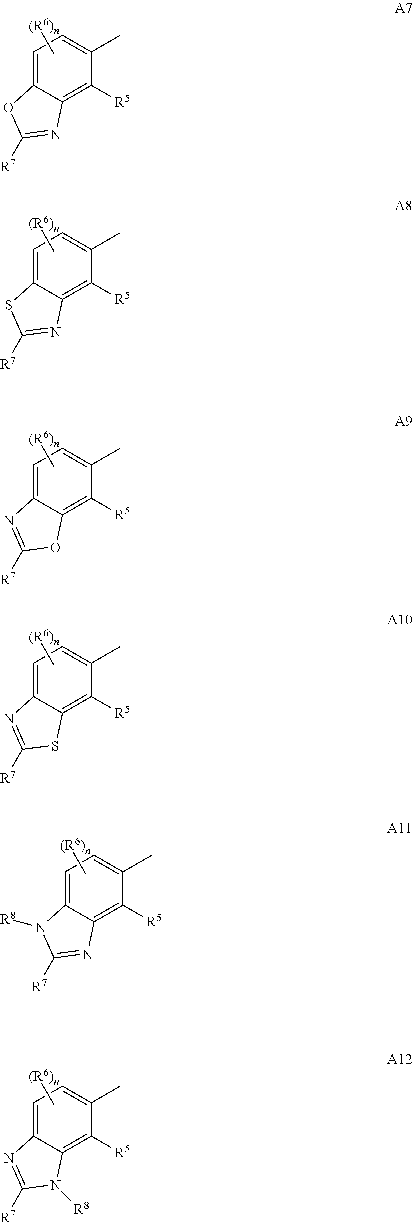

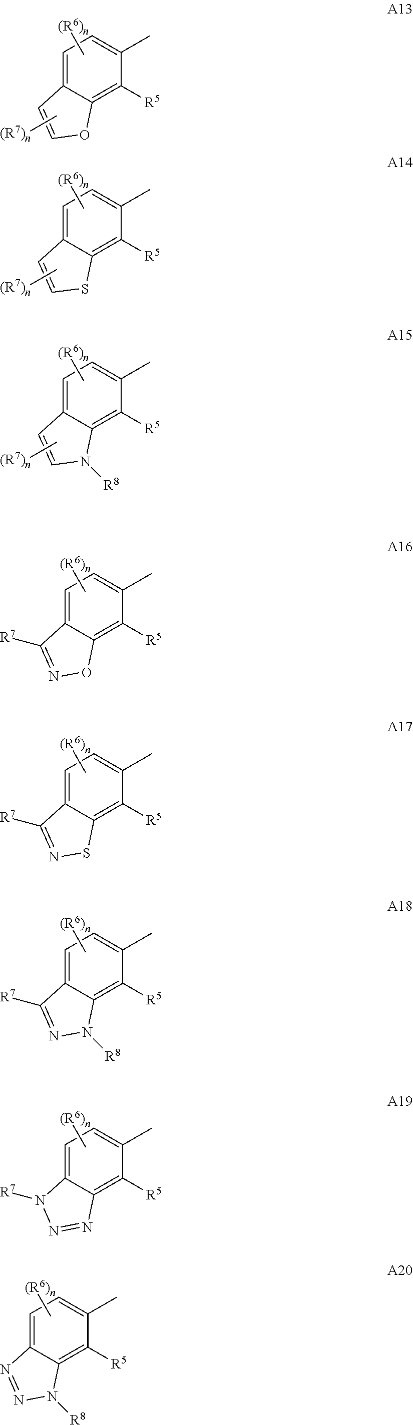

R¹ represents halogen, CN, NO₂, OH, NH₂, (C₁-C₆)-alkyl, (C₁-C₆)-alkoxy, (C₁-C₆)-haloalkyl, (C₁-C₆)-haloalkoxy, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, (C₁-C₆)-alkylcarbonyl, (C₁-C₆)-alkylcarboxy, (C₁-C₆)-alkylamine, di(C₁-C₆)-alkylamine, (C₁-C₆)-alkyl-S(O)ₙ or (C₁-C₆)-alkyl-S(O)₂NH,
R² represents chlorine or fluorine,
R³ represents hydrogen,
R⁴ represents hydrogen,
R⁵ represents hydrogen, halogen, OH, NH₂, CN, (C₁-C₃)-alkyl, (C₁-C₃)-alkoxy, C₁-C₃)-alkylamino or cyclopropyl,
R⁶ represents hydrogen, halogen, OH, NH₂, CN, (C₁-C₃)-alkyl, (C₁-C₃)-alkoxy, cyclopropyl or vinyl,
R⁷ represents hydrogen, halogen, (C₁-C₃)-alkyl, (C₁-C₃)-alkoxy, (C₁-C₃)-alkylthio, cyclopropyl, (C₁-C₃)-alkylamino or phenyl,
R⁸ represents hydrogen, (C₁-C₆)-alkyl, (C₁-C₄)-alkylcarbonyl, (C₁-C₆)-alkoxycarbonyl or phenyl,
X represents CH or CF,
m represents 1, 2, 3, 4 or 5 and
n represents 0, 1 or 2.

A first embodiment of the present invention encompasses compounds of the general formula (I) in which
A is preferably selected from the group consisting of A1 to A3, A7 to A15 and A17 to A18,

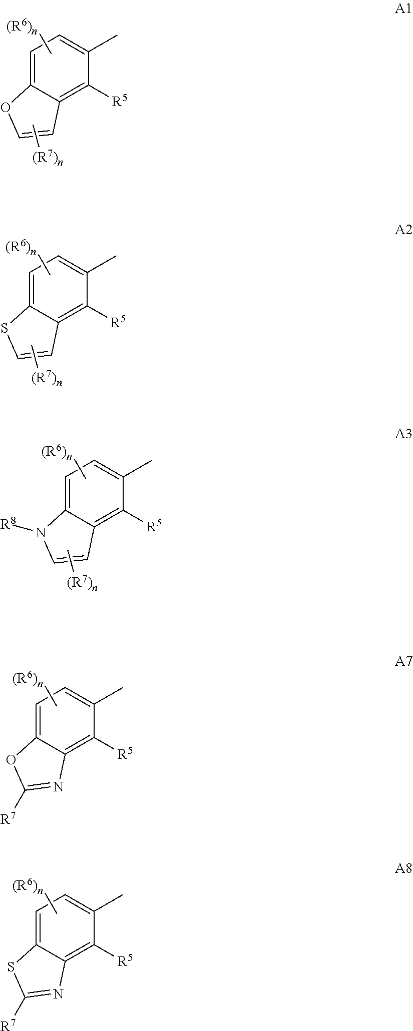

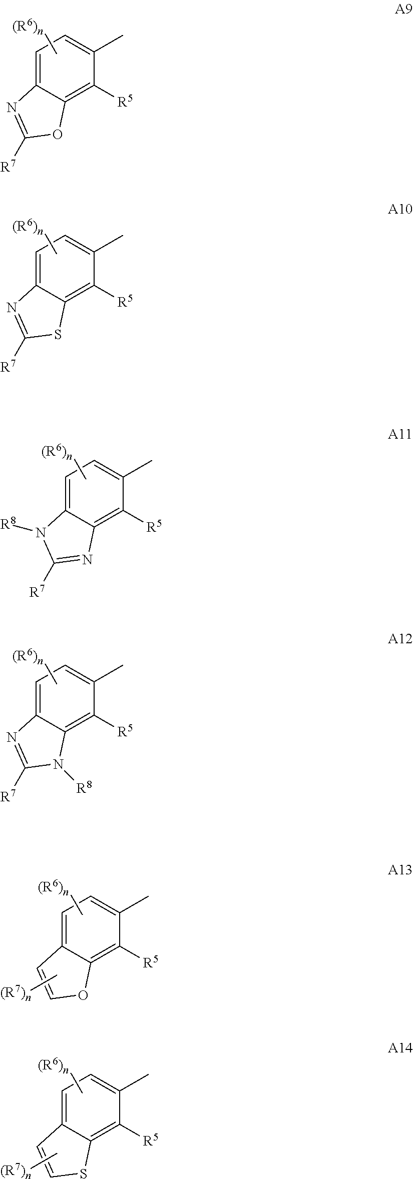

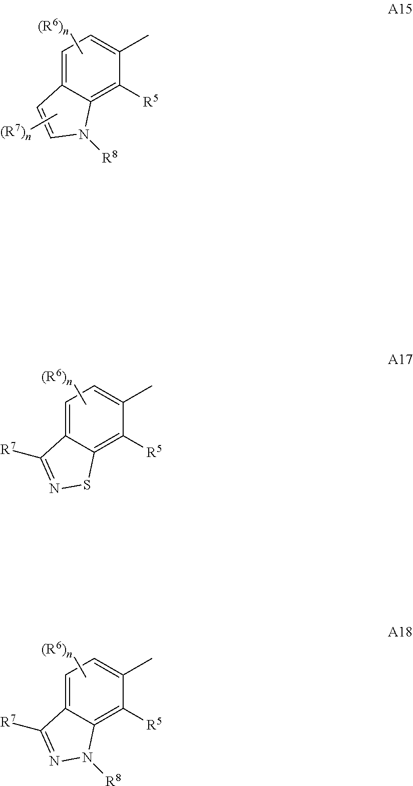

A is particularly preferably selected from the group consisting of the radicals A1 to A3 and A13 to A15;
A most preferably represents A2 or A15.

A second embodiment of the present invention encompasses compounds of the general formula (I) in which
R¹ preferably represents halogen, CN, NO₂, OH, NH₂, (C₁-C₆)-alkyl, (C₁-C₆)-alkoxy, (C₁-C₆)-haloalkyl or (C₁-C₆)-haloalkoxy, in particular halogen and most preferably fluorine.

A third embodiment of the present invention encompasses compounds of the general formula (I) in which
R² preferably represents chlorine.

A fourth embodiment of the present invention encompasses compounds of the general formula (I) in which
R³ preferably represents hydrogen.

A fifth embodiment of the present invention encompasses compounds of the general formula (I) in which
R⁴ preferably represents hydrogen.

A sixth embodiment of the present invention encompasses compounds of the general formula (I) in which
R⁵ preferably represents hydrogen or halogen and particularly preferably hydrogen or fluorine.

A seventh embodiment of the present invention encompasses compounds of the general formula (I) in which
R⁶ preferably represents hydrogen or halogen and particularly preferably hydrogen.

An eighth embodiment of the present invention encompasses compounds of the general formula (I) in which
R⁷ preferably represents hydrogen, halogen or (C₁-C₃)-alkyl and particularly preferably hydrogen.

A ninth embodiment of the present invention encompasses compounds of the general formula (I) in which
R⁸ preferably represents hydrogen, (C₁-C₄)-alkyl, (C₁-C₄)-alkylcarbonyl or (C₁-C₄)-alkoxycarbonyl, particularly preferably hydrogen, (C₁-C₃)-alkyl, (C₁-C₄)-alkylcarbonyl or (C₁-C₄)-alkoxycarbonyl and most preferably hydrogen.

A tenth embodiment of the present invention encompasses compounds of the general formula (I) in which
X preferably represents CH or CF.

An eleventh embodiment of the present invention encompasses compounds of the general formula (I) in which
m preferably represents 1, 2 or 3, particularly preferably 1 or 2 and most preferably 1.

A twelfth embodiment of the present invention encompasses compounds of the general formula (I) in which
n preferably represents 0 or 1 and particularly preferably 0.

In the context of the present invention, the individual preferred, particularly preferred and very particularly preferred meanings of the substituents A, R¹ to R⁸ and X may be combined with one another as desired, where the running number n represents 0, 1 or 2, preferably 0 or 1 and very particularly preferably 0 and the running number m represents 1, 2, 3, 4 or 5, preferably 1, 2 or 3, particularly preferably 1 or 2 and most preferably 1.

This means that the present invention encompasses compounds of the general formula (I) in which, for example, the radical A has a preferred meaning and the substituents R¹ to R⁷ have the general definition or else the substituent R² has a preferred meaning, the substituent R⁴ has a particularly preferred meaning and the remaining substituents have a general meaning.

Three of these combinations of the definitions given above for the substituents A, R¹ to R⁸ and X and for the running numbers n and m are elucidated by way of example hereinafter and each are explicitly disclosed as further embodiments for reasons of clarity:

A thirteenth embodiment of the present invention encompasses compounds of the general formula (I) in which
A is selected from A1 to A3 and A13 to A15,
R¹ represents halogen, CN, NO₂, OH, NH₂, (C₁-C₆)-alkyl, (C₁-C₆)-alkoxy, (C₁-C₆)-haloalkyl or (C₁-C₆)-haloalkoxy,
R² represents chlorine,
R³ represents hydrogen
R⁴ represents hydrogen
R⁵ represents hydrogen or fluorine,
R⁶ represents hydrogen or halogen,
R⁷ represents hydrogen, halogen or (C₁-C₃)-alkyl,
R⁸ represents hydrogen, (C₁-C₄)-alkyl, (C₁-C₄)-alkylcarbonyl or (C₁-C₄)-alkoxycarbonyl,
X represents CH or CF,
n represents 0 or 1 and
m represents 1, 2 or 3.

A fourteenth embodiment of the present invention encompasses compounds of the general formula (I) in which
A is selected from A1 to A3 and A13 to A15,
R¹ represents halogen,
R² represents chlorine,
R³ represents hydrogen
R⁴ represents hydrogen
R⁵ represents hydrogen or fluorine,
R⁶ represents hydrogen
R⁷ represents hydrogen
R⁸ represents hydrogen, (C₁-C₃)-alkyl, (C₁-C₄)-alkylcarbonyl or (C₁-C₄)-alkoxycarbonyl,
X represents CH or CF,
n represents 0 or 1 and
m represents 1 or 2.

A fifteenth embodiment of the present invention encompasses compounds of the general formula (I) in which
A represents A2 or A15,
R¹ represents fluorine,
R² represents chlorine,
R³ represents hydrogen
R⁴ represents hydrogen
R⁵ represents hydrogen or fluorine,
R⁶ represents hydrogen
R⁷ represents hydrogen
R⁸ represents hydrogen, (C₁-C₃)-alkyl, (C₁-C₄)-alkylcarbonyl or (C₁-C₄)-alkoxycarbonyl,
X represents CH or CF,
n represents 0 or 1 and
m represents 1.

Alkylcarbonyl (alkyl-C(═O)—) represents saturated straight-chain or branched alkyl radicals attached to the skeleton via —C(═O)—, such as (C₁-C₁₀)—, (C₁-C₆)- or (C₁-C₄)-alkylcarbonyl. Here, the number of the carbon atoms refers to the alkyl radical in the alkylcarbonyl group.

Alkyl represents saturated straight-chain or branched hydrocarbyl radicals having 1 to 10 carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Haloalkyl represents straight-chain or branched alkyl groups having 1 to 8 carbon atoms, where in these groups some or all of the hydrogen atoms may be replaced by halogen atoms, for example C₁-C₂-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl.

Alkenyl represents unsaturated straight-chain or branched hydrocarbyl radicals having 2 to 8 carbon atoms and one double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, I-methyl-2-propenyl, 2-methyl-2-propenyl, 1-pentenyl, 2-pentenyl, 3-pentenyl, 4-pentenyl, i-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, i-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

Alkynyl represents straight-chain or branched hydrocarbyl radicals having 2 to 8 carbon atoms and one triple bond in any position, for example C₂-C₆-alkynyl such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, i-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

Alkoxy represents saturated straight-chain or branched alkoxy radicals having 1 to 8 carbon atoms, for example C₁-C₆-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;

Haloalkoxy represents straight-chain or branched alkoxy groups having 1 to 8 carbon atoms (as mentioned above), where in these groups some or all of the hydrogen atoms may be replaced by halogen atoms as mentioned above, for example C₁-C₂-haloalkoxy such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy and 1,1,1-trifluoroprop-2-oxy.

Alkylthio represents saturated, straight-chain or branched alkylthio radicals having 1 to 8 carbon atoms, for example C₁-C₆-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;

Haloalkylthio represents straight-chain or branched alkylthio groups having 1 to 8 carbon atoms (as mentioned above), where in these groups some or all of the hydrogen atoms may be replaced by halogen atoms as mentioned above, for example C₁-C₂-haloalkylthio such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio and 1,1,1-trifluoroprop-2-ylthio.

Aryl is phenyl or naphthyl.

Depending inter alia on the nature of the substituents, the compounds of the formula (I) can be present as geometrical and/or optical isomers or isomer mixtures of varying composition which, if desired, can be separated in a customary manner. The present invention provides both the pure isomers and the isomer mixtures, their preparation and use and compositions comprising them. However, the following text will, for the sake of simplicity, always mention compounds of the formula (I), even though this is understood as meaning not only the pure compounds, but also, if appropriate, mixtures with various amounts of isomeric compounds.

A metal ion equivalent is a metal ion having a positive charge, such as Na⁺, K⁺, (Mg²⁺)_{1/2}, (Ca²⁺)_{1/2}, MgH⁺, CaH⁺, (Al³⁺)_{1/3} (Fe²⁺)_{1/2} or (Fe⁺)_{1/3}.

Halogen is fluorine, chlorine, bromine and iodine.

If a group is polysubstituted by radicals, this should be understood to mean that this group is substituted by one or more identical or different radicals selected from the radicals mentioned.

Depending on the nature of the substituents defined above, the compounds of the formula (I) have acidic or basic properties and can form salts, if appropriate also inner salts, or adducts with inorganic or organic acids or with bases or with metal ions. If the compounds of the formula (I) carry amino, alkylamino or other groups which induce basic properties, these compounds can be reacted with acids to give salts, or they are directly obtained as salts in the synthesis.

Examples of inorganic acids are hydrohalic acids such as hydrogen fluoride, hydrogen chloride, hydrogen bromide and hydrogen iodide, sulfuric acid, phosphoric acid and nitric acid, and acidic salts such as NaHSO₄ und KHSO₄. Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulfonic acids (sulfonic acids having straight-chain or branched alkyl radicals of 1 to 20 carbon atoms), arylsulfonic acids or aryldisulfonic acids (aromatic radicals, such as phenyl and naphthyl, which carry one or two sulfonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulfonic acid, salicylic acid, p-aminosalicylic acid, 2-phenoxybenzoic acid, 2-acetoxybenzoic acid, etc.

Useful metal ions are especially the ions of the elements of the second main group, especially calcium and magnesium, of the third and fourth main groups, especially aluminum, tin and lead, and also of the first to eighth transition groups, especially chromium, manganese, iron, cobalt, nickel, copper, zinc and others. Particular preference is given to the metal ions of the elements of the fourth period. The metals may be present in the different valences that they can assume.

If the compounds of the formula (I) bear hydroxyl, carboxyl or other groups which induce acidic properties, these compounds can be reacted with bases to give salts. Suitable bases are, for example, hydroxides, carbonates, hydrogencarbonates of the alkali metals and alkaline earth metals, especially those of sodium, potassium, magnesium and calcium, and also ammonia, primary, secondary and tertiary amines having (C₁-C₄)-alkyl groups, mono-, di- and trialkanolamines of (C₁-C₄)-alkanols, choline and chlorocholine.

Depending on the nature of the substituents and the manner in which they are attached, the compounds of the general formula (I) may be present as stereoisomers. If, for example, one or more asymmetrically substituted carbon atoms or sulfoxides are present, enantiomers and diastereomers may occur.

Stereoisomers can be obtained from the mixtures obtained in the preparation by customary separation methods, for example by chromatographic separation processes. It is likewise possible to selectively prepare stereoisomers by using stereoselective reactions with use of optically active starting materials and/or auxiliaries. The invention also relates to all stereoisomers and mixtures thereof which are encompassed by the general formula (I) but not defined specifically.

In all the formulae specified hereinafter, the substituents and symbols have the same meaning as described in formula (I), unless defined differently.

Compounds of the formula (I) can be prepared, for example, according to the scheme below by base-catalyzed esterification of a carboxylic acid of the formula II with a benzyl bromide of the formula III.

In this scheme, Het represents the heterocycles of groups A1 to A24 condensed to the phenyl ring.

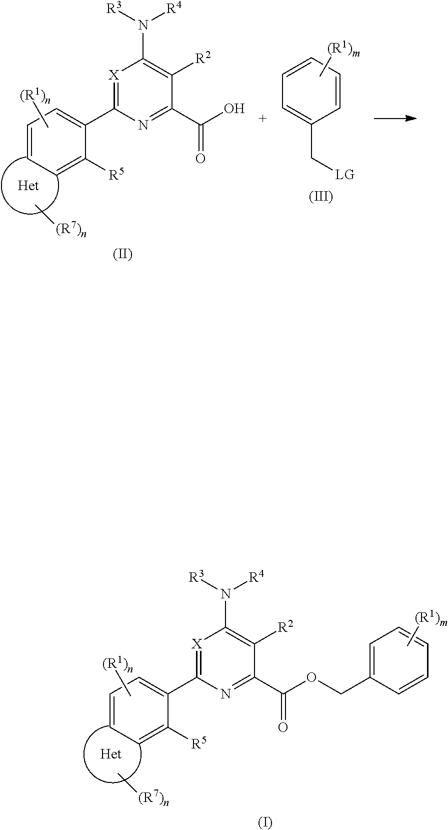

The carboxylic acids of the formula (II) are known, for example, from WO2013/14165 A1, or they can be prepared by methods known per se to the person skilled in the art. The benzyl derivatives of the formula (III) are commercially available or can be prepared by methods known per se to the person skilled in the art.

Preference is given to the compounds of the formula (I) listed in the table I below,

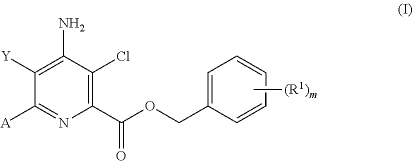

where X═C—Y, R²═Cl, R³═R⁴═H

TABLE 1

| Number | A | Y | Phenyl-(R¹)ₘ |
|---|---|---|---|
| I-01 | 1-benzothiophen-6-yl | H | 2-fluorophenyl |
| I-02 | 1-benzofuran-6-yl | H | 3-fluorophenyl |
| I-03 | 1-benzofuran-6-yl | F | 4-methoxyphenyl |
| I-04 | 1-benzofuran-6-yl | F | 4-fluorophenyl |
| I-05 | 1-benzofuran-5-yl | H | 4-fluorophenyl |
| I-06 | 1-benzofuran-5-yl | H | 2,4,5-trifluorophenyl |
| I-07 | 1-benzothiophen-6-yl | H | 4-fluorophenyl |
| I-08 | 1-benzothiophen-6-yl | H | 2,4-difluoro-3- |
| | | | methoxyphenyl |
| I-09 | 1-benzothiophen-5-yl | H | 3-fluorophenyl |
| I-10 | 1-benzothiophen-6-yl | H | 3-fluorophenyl |
| I-11 | 1-benzofuran-5-yl | H | 3-(trifluoromethyl)phenyl |
| I-12 | 1-benzothiophen-6-yl | H | 2,4-difluorophenyl |
| I-13 | 1-benzofuran-5-yl | H | 2,3,4-trifluorophenyl |
| I-14 | 1-benzothiophen-6-yl | H | 4-chlorophenyl |
| I-15 | 1-benzofuran-6-yl | F | 4-chlorophenyl |
| I-16 | 1-benzothiophen-5-yl | H | 4-(trifluoromethyl)phenyl |
| I-17 | 1-benzofuran-5-yl | H | 2,4-difluoro-3- |
| | | | methoxyphenyl |
| I-18 | 1-benzothiophen-5-yl | H | 2,4-difluorophenyl |
| I-19 | 1-benzothiophen-5-yl | H | 4-chlorophenyl |
| I-20 | 1-benzofuran-5-yl | H | 2,4,6-trifluorophenyl |
| I-21 | 1-benzothiophen-5-yl | H | 3-(trifluoromethyl)phenyl |
| I-22 | 1-benzothiophen-5-yl | H | 2,4-difluoro-3- |
| | | | methoxyphenyl |
| I-23 | 1-benzofuran-6-yl | H | 2,3,4-trifluorophenyl |
| I-24 | 1-benzofuran-5-yl | H | 2,4-difluorophenyl |
| I-25 | 1-benzothiophen-6-yl | H | 2,3,4,5,6-pentafluorophenyl |
| I-26 | 1-benzofuran-6-yl | H | 4-fluorophenyl |
| I-27 | 1-benzofuran-6-yl | H | 2,4,5-trifluorophenyl |
| I-28 | 1-benzofuran-6-yl | H | 2,4-difluoro-3- |
| | | | methoxyphenyl |
| I-29 | 1-benzothiophen-5-yl | F | 4-fluorophenyl |
| I-30 | 1-benzofuran-6-yl | H | 4-chlorophenyl |
| I-31 | 1-benzofuran-5-yl | H | 4-chlorophenyl |
| I-32 | 1-benzothiophen-6-yl | H | 2,4,6-trifluorophenyl |
| I-33 | 7-fluoro-1-benzofuran- | H | 3-chlorophenyl |
| | 6-yl | | |
| I-34 | 1-benzofuran-5-yl | F | 4-fluorophenyl |
| I-35 | 1-benzofuran-6-yl | F | 2,4-dimethoxyphenyl |
| I-36 | 1-benzofuran-5-yl | H | 4-(trifluoromethyl)phenyl |
| I-37 | 1-benzofuran-5-yl | H | 2-(trifluoromethyl)phenyl |
| I-38 | 1-benzofuran-5-yl | H | 3-chlorophenyl |
| I-39 | 1,3-benzothiazol-6-yl | H | 2,4-difluorophenyl |
| I-40 | 1-benzothiophen-5-yl | H | 3-chlorophenyl |
| I-41 | 1,3-benzothiazol-6-yl | H | 3-fluorophenyl |
| I-42 | 1-benzothiophen-6-yl | F | 2,4-difluorophenyl |
| I-43 | 1-benzofuran-6-yl | H | 2,4-difluorophenyl |
| I-44 | 1-benzothiophen-6-yl | H | 2-chlorophenyl |
| I-45 | 1-benzothiophen-6-yl | F | 4-chlorophenyl |
| I-46 | 1-benzothiophen-6-yl | H | 2,3,4-trifluorophenyl |
| I-47 | 1-benzofuran-5-yl | F | 4-chlorophenyl |
| I-48 | 1-benzofuran-5-yl | F | 2,4-difluorophenyl |
| I-49 | 7-fluoro-1-benzofuran- | H | 4-chlorophenyl |
| | 6-yl | | |
| I-50 | 1-benzothiophen-6-yl | H | 3-chlorophenyl |
| I-51 | 1-benzofuran-6-yl | F | 2,4-dichlorophenyl |
| I-52 | 1-benzofuran-5-yl | F | 2,4,6-trifluorophenyl |
| I-53 | 1-benzothiophen-6-yl | F | 4-fluorophenyl |
| I-54 | 1-benzofuran-6-yl | H | 2,3,4,5,6-pentafluorophenyl |
| I-55 | 1-benzothiophen-6-yl | F | 2,4,6-trifluorophenyl |
| I-56 | 1-benzothiophen-5-yl | F | 2,4-difluorophenyl |
| I-57 | 1-benzofuran-5-yl | H | 3,6-dichloro-2- |
| | | | methoxyphenyl |
| I-58 | 6-fluoro-1- | F | 4-chlorophenyl |
| | benzothiophen-5-yl | | |
| I-59 | 1-benzothiophen-5-yl | F | 2,4,6-trifluorophenyl |
| I-60 | 1,3-benzothiazol-6-yl | H | 3-chlorophenyl |
| I-61 | 1-benzothiophen-6-yl | H | 2,3,4,5-tetrafluorophenyl |
| I-62 | 1-benzofuran-5-yl | H | 2-nitrophenyl |
| I-63 | 1-benzofuran-5-yl | F | 2,4-dichlorophenyl |
| I-64 | 1,3-benzothiazol-6-yl | F | 4-chlorophenyl |
| I-65 | 1-benzothiophen-5-yl | F | 4-chlorophenyl |
| I-66 | 6-fluoro-1-benzofuran- | F | 2,4-difluorophenyl |
| | 5-yl | | |
| I-67 | 1-benzofuran-6-yl | H | 2,3,4,5-tetrafluorophenyl |
| I-68 | 6-fluoro-1-benzofuran- | F | 4-chlorophenyl |
| | 5-yl | | |
| I-69 | 1,3-benzothiazol-6-yl | H | 4-chlorophenyl |
| I-70 | 1,3-benzothiazol-6-yl | F | 3-fluorophenyl |
| I-71 | 6-fluoro-1-benzofuran- | F | 4-fluorophenyl |
| | 5-yl | | |
| I-72 | 1,3-benzothiazol-6-yl | H | 4-(trifluoromethyl)phenyl |
| I-73 | 1-benzothiophen-5-yl | H | 2,3,4,5-tetrafluorophenyl |
| I-74 | 1-benzofuran-5-yl | H | 2,3,4,5-tetrafluorophenyl |
| I-75 | 6-fluoro-1- | F | 2,4-difluorophenyl |
| | benzothiophen-5-yl | | |
| I-76 | 1-benzothiophen-6-yl | F | 2,4-dichlorophenyl |
| I-77 | 6-fluoro-1- | F | 4-fluorophenyl |
| | benzothiophen-5-yl | | |
| I-78 | 1,3-benzothiazol-6-yl | H | 2,4-dichlorophenyl |
| I-79 | 6-fluoro-1-benzofuran- | F | 2,4,6-trifluorophenyl |
| | 5-yl | | |
| I-80 | 6-fluoro-1- | F | 2,4-dichlorophenyl |
| | benzothiophen-5-yl | | |
| I-81 | 6-fluoro-1-benzofuran- | F | 2,4-dichlorophenyl |
| | 5-yl | | |
| I-82 | 1-benzothiophen-5-yl | F | 2,4-dichlorophenyl |
| I-83 | 1,3-benzothiazol-6-yl | F | 4-fluorophenyl |
| I-84 | 7-fluoro-1H-indol-6-yl | F | 3-fluorophenyl |

Collections of compounds of the formula (I) and/or salts thereof which can be synthesized by the abovementioned reactions can also be prepared in a parallelized manner, in which case this may be accomplished in a manual, partly automated or fully automated manner. It is possible, for example, to automate the conduct of the reaction, the workup or the purification of the products and/or intermediates. Overall, this is understood to mean a procedure as described, for example, by D. Tiebes in Combinatorial Chemistry—Synthesis, Analysis, Screening (editor: Günther Jung), Wiley, 1999, on pages 1 to 34.

For the parallelized conduct of the reaction and workup, it is possible to use a number of commercially available instruments, for example Calypso reaction blocks from Barnstead International, Dubuque, Iowa 52004-0797, USA or reaction stations from Radleys, Shirehill, Saffron Walden, Essex, CB 11 3AZ, England, or MultiPROBE Automated Workstations from Perkin Elmer, Waltham, Mass. 02451, USA. For the parallelized purification of compounds of the formula (I) and salts thereof or of intermediates which occur in the course of preparation, available apparatuses include chromatography apparatuses, for example from ISCO, Inc., 4700 Superior Street, Lincoln, Nebr. 68504, USA.

The apparatuses detailed lead to a modular procedure in which the individual working steps are automated, but manual operations have to be carried out between the working steps. This can be circumvented by using partly or fully integrated automation systems in which the respective automation modules are operated, for example, by robots. Automation systems of this type can be obtained, for example, from Caliper, Hopkinton, Mass. 01748, USA.

The implementation of single or multiple synthesis steps can be supported by the use of polymer-supported reagents/scavenger resins. The specialist literature describes a series of experimental protocols, for example in ChemFiles, Vol. 4, No. 1, Polymer-Supported Scavengers and Reagents for Solution-Phase Synthesis (Sigma-Aldrich).

Aside from the methods described here, the compounds of the formula (I) and salts thereof can be prepared completely or partially by solid-phase supported methods. For this purpose, individual intermediates or all intermediates in the synthesis or a synthesis adapted for the corresponding procedure are bound to a synthesis resin. Solid-phase-supported synthesis methods are described adequately in the technical literature, for example Barry A. Bunin in “The Combinatorial Index”, Academic Press, 1998 and Combinatorial Chemistry—Synthesis, Analysis, Screening (editor: Günther Jung), Wiley, 1999. The use of solid-phase-supported synthesis methods permits a number of protocols, which are known from the literature and which for their part may be performed manually or in an automated manner. The reactions can be performed, for example, by means of IRORI technology in microreactors from Nexus Biosystems, 12140 Community Road, Poway, Calif. 92064, USA.

Both in the solid and in the liquid phase, the implementation of individual or several synthesis steps may be supported by the use of microwave technology. The specialist literature describes a series of experimental protocols, for example in Microwaves in Organic and Medicinal Chemistry (editor: C. O. Kappe and A. Stadler), Wiley, 2005.

The preparation by the processes described here gives compounds of the formula (I) and salts thereof in the form of substance collections, which are called libraries. The present invention also provides libraries comprising at least two compounds of the formula (I) and salts thereof.

The compounds of the formula (I) according to the invention (and/or salts thereof), referred to collectively as “compounds according to the invention” hereinafter, have excellent herbicidal efficacy against a broad spectrum of economically important monocotyledonous and dicotyledonous annual harmful plants. The active ingredients also have good control over perennial harmful plants which are difficult to control and produce shoots from rhizomes, root stocks or other perennial organs.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

If the compounds of the invention are applied to the soil surface before germination, either the emergence of the weed seedlings is prevented completely or the weeds grow until they have reached the cotyledon stage, but then they stop growing and ultimately die completely after three to four weeks have passed.

If the active compounds are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the compounds of the invention have outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, will be damaged to a negligible extent only, if at all, depending on the structure of the particular compound of the invention and its application rate. For these reasons, the present compounds are very suitable for selective control of unwanted plant growth in plant crops such as agriculturally useful plants or ornamental plants.

In addition, the compounds of the invention (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the active compounds can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the inventive compounds of the formula (I) or salts thereof in economically important transgenic crops of useful plants and ornamental plants, for example of cereals such as wheat, barley, rye, oats, millet, rice, cassava and corn, or else crops of sugar beet, cotton, soybean, oilseed rape, potatoes, tomatoes, peas and other vegetables.

The compounds of the formula (I) can preferably be used as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants.

Alternatively, novel plants with altered properties can be generated with the aid of recombinant methods (see, for example, EP 0221044, EP 0131624). For example, there have been descriptions in several cases of:
genetic modifications of crop plants for the purpose of modifying the starch synthesized in the plants (for example WO 92/011376 A, WO 92/014827 A, WO 91/019806 A),
transgenic crop plants which are resistant to certain herbicides of the glufosinate type (cf., for example, EP 0242236 A, EP 0242246 A) or of the glyphosate type (WO 92/000377A) or of the sulfonylurea type (EP 0257993 A, U.S. Pat. No. 5,013,659) or to combinations or mixtures of these herbicides through “gene stacking”, such as transgenic crop plants, for example corn or soya with the trade name or the designation Optimum™ GAT™ (Glyphosate ALS Tolerant).
transgenic crop plants, for example cotton, capable of producing Bacillus thuringiensis toxins (Bt toxins), which make the plants resistant to particular pests (EP-A-0142924, EP-A-0193259).
transgenic crop plants having a modified fatty acid composition (WO 91/013972 A).
genetically modified crop plants having novel constituents or secondary metabolites, for example novel phytoalexins, which cause an increase in disease resistance (EP 0309862 A, EP 0464461 A)
genetically modified plants having reduced photorespiration, which have higher yields and higher stress tolerance (EP 0305398 A)
transgenic crop plants which produce pharmaceutically or diagnostically important proteins (“molecular pharming”)
transgenic crop plants which feature higher yields or better quality
transgenic crop plants which are distinguished by a combination, for example of the abovementioned novel properties (“gene stacking”)

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such recombinant manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To join the DNA fragments with one another, adapters or linkers can be placed onto the fragments, see, for example, Sambrook et al., 1989, Molecular Cloning, A Laboratory Manual, 2nd edition Cold Spring Harbor Laboratory Press, Cold Spring Harbor, N.Y., or Winnacker “Gene und Klone” [Genes and clones], VCH Weinheim 2nd edition 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Thus, transgenic plants can be obtained whose properties are altered by overexpression, suppression or inhibition of homologous (=natural) genes or gene sequences or expression of heterologous (=foreign) genes or gene sequences.

The compounds (I) according to the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active compounds, or to any desired combinations of these active compounds.

The compounds of the invention can be used with particular preference in transgenic crop plants which are resistant to a combination of glyphosates and glufosinates, glyphosates and sulfonylureas or imidazolinones. The compounds of the invention can be used with very particular preference in transgenic crop plants, for example corn or soybeans with the trade name or the designation Optimum™ GAT™ (glyphosate ALS tolerant).

When the active compounds of the invention are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types are known in principle and are described, for example, in: Winnacker-Küchler, “Chemische Technologie” [Chemical Engineering],

volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J.; H.v. Olphen, “Introduction to Clay Colloid Chemistry”, 2nd Ed., J. Wiley & Sons, N.Y.; C. Marsden, “Solvents Guide”, 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. Publ. Co. Inc., N.Y. 1964; SchSnfeldt, “Grenzflächenaktive Äthylenoxidaddukte” [Interface-active Ethylene Oxide Adducts], Wiss. Verlagsgesellschaft, Stuttgart 1976; Winnacker-Küchler, “Chemische Technologie” [Chemical Engineering], volume 7, C. Hanser Verlag Munich, 4th Ed. 1986.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix.

Suitable safeners are, for example, mefenpyr-diethyl, cyprosulfamide, isoxadifen-ethyl, cloquintocet-mexyl and dichlormid.

The safeners are preferably selected from the group consisting of:
S1) Compounds of the formula (S1)

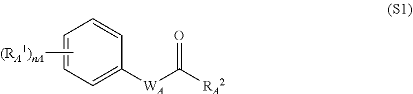

where the symbols and indices are defined as follows:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group of the partially unsaturated or aromatic five-membered heterocycles having 1 to 3 ring heteroatoms from the N and O group, where at least one nitrogen atom and at most one oxygen atom is present in the ring, preferably a radical from the group of (W_{A}¹) to (W_{A}⁴),
m_{A} is 0 or I;

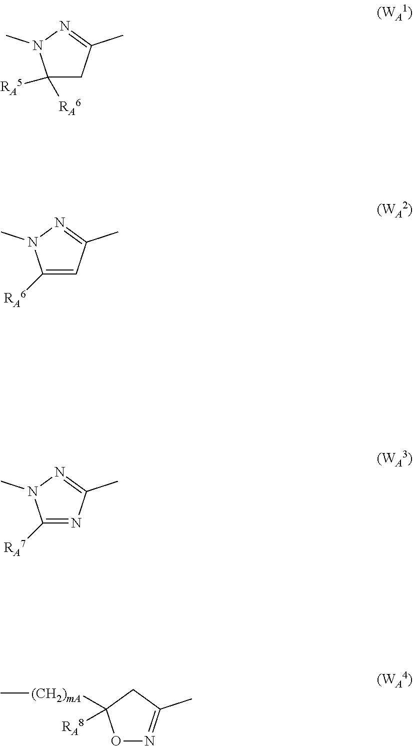

R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is joined to the carbonyl group in (S1) via the nitrogen atom and is unsubstituted or substituted by radicals from the group consisting of (C₁-C₄)-alkyl, (C₁-C₄)-alkoxy or optionally substituted phenyl, preferably a radical of the formula OR_{A}³, NHR_{A}⁴ or N(CH₃)₂, especially of the formula OR_{A}³;
R_{A}³ is hydrogen or an unsubstituted or substituted aliphatic hydrocarbon radical preferably having a total of 1 to 18 carbon atoms;
R_{A}⁴ is hydrogen, (C₁-C₆)-alkyl, (C₁-C₆)-alkoxy or substituted or unsubstituted phenyl;
R_{A}⁵ is H, (C₁-C₆)-alkyl, (C₁-C₆)-haloalkyl, (C₁-C₄)-alkoxy-(C₁-C₈)-alkyl, cyano or COOR_{A}⁹, where R_{A}⁹ is hydrogen, (C₁-C₆)-alkyl, (C₁-C₆)-haloalkyl, (C₁-C₄)-alkoxy-(C₁-C₄)-alkyl, (C₁-C₆)-hydroxyalkyl, (C₃-C₁₂)-cycloalkyl or tri-(C₁-C₄)-alkylsilyl;
R_{A}⁶, R_{A}⁷, R_{A}⁸ are identical or different and are each hydrogen, (C₁-C₆)-alkyl, (C₁-C₆)-haloalkyl, (C₃-C₁₂)-cycloalkyl or substituted or unsubstituted phenyl;
preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333 131 and EP-A-269 806;
c) derivatives of 1,5-diphenylpyrazole-3-carboxylic acid (S1^{c}), preferably compounds such as ethyl I-(2,4-dichlorophenyl)-5-phenylpyrazole-3-carboxylate (S1-5), methyl I-(2-chlorophenyl)-5-phenylpyrazole-3-carboxylate (S1-6) and related compounds as described in EP-A-268 554, for example;
d) compounds of the triazolecarboxylic acid type (S1^{d}), preferably compounds such as fenchlorazole(-ethyl ester), i.e. ethyl I-(2,4-dichlorophenyl)-5-trichloromethyl-(H)-1,2,4-triazole-3-carboxylate (S1-7), and related compounds as described in EP-A-174 562 and EP-A-346 620;
e) compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazoline-3-carboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13), as described in patent application WO-A-95/07897.
S2) Quinoline derivatives of the formula (S2)

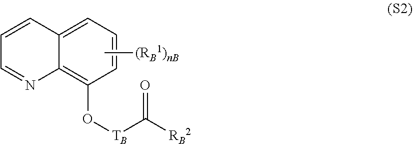

where the symbols and indices have the meanings below:
R_{B}¹ is halogen, (C₁-C₄)-alkyl, (C₁-C₄)-alkoxy, nitro or (C₁-C₄)-haloalkyl;
n_{B} is a natural number from 0 to 5, preferably from 0 to 3;
R_{B}² is OR_{B}³, SR_{B}³ or NR_{B}³R_{B}⁴ or a saturated
   or unsaturated 3- to 7-membered heterocycle having at least one nitrogen atom and up to 3 heteroatoms, preferably from the group of O and S, which is joined via the nitrogen atom to the carbonyl group in (S2) and is unsubstituted or substituted by radicals from the group of (C₁-C₄)-alkyl, (C₁-C₄)-alkoxy or optionally substituted phenyl, preferably a radical of the formula OR_{B}³, NHR_{B}⁴ or N(CH₃)₂, especially of the formula OR_{B}³;
R_{B}³ is hydrogen or an unsubstituted or substituted aliphatic hydrocarbon radical preferably having a total of 1 to 18 carbon atoms;
R_{B}⁴ is hydrogen, (C₁-C₆)-alkyl, (C₁-C₆)-alkoxy or substituted or unsubstituted phenyl;
T_{B} is a (C₁ or C₂)-alkanediyl chain which is unsubstituted or substituted by one or two (C₁-C₄)-alkyl radicals or by [(C₁-C₃)-alkoxy]carbonyl;
preferably:
a) compounds of the 8-quinolinoxyacetic acid type (S2^{a}), preferably
   1-methylhexyl (5-chloro-8-quinolinoxy)acetate (“cloquintocet-mexyl”) (S2-1),
   1,3-dimethylbut-1-yl (5-chloro-8-quinolinoxy)acetate (S2-2),
   4-allyloxybutyl (5-chloro-8-quinolinoxy)acetate (S2-3),
   1-allyloxyprop-2-yl (5-chloro-8-quinolinoxy)acetate (S2-4),
   ethyl (5-chloro-8-quinolinoxy)acetate (S2-5),
   methyl 5-chloro-8-quinolinoxyacetate (S2-6),
   allyl (5-chloro-8-quinolinoxy)acetate (S2-7),
   2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
b) compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Compounds of the formula (S3)

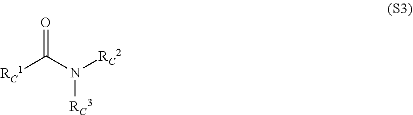

where the symbols and indices are defined as follows:
R_{C}¹ is (C₁-C₄)-alkyl, (C₁-C₄)-haloalkyl, (C₂-C₄)-alkenyl, (C₂-C₄)-haloalkenyl, (C₃-C₇)-cycloalkyl, preferably dichloromethyl;
R_{C}², R_{C}³ are identical or different and are hydrogen, (C₁-C₄)-alkyl, (C₂-C₄)-alkenyl, (C₂-C₄)-alkynyl, (C₂-C₄)-haloalkyl, (C₂-C₄)-haloalkenyl, (C₁-C₄)-alkylcarbamoyl-(C₁-C₄)-alkyl, (C₂-C₄)-alkenylcarbamoyl-(C₁-C₄)-alkyl, (C₁-C₄)-alkoxy-(C₁-C₄)-alkyl, dioxolanyl-(C₁-C₄)-alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
preferably:
   Active compounds of the dichloroacetamide type, which are frequently used as pre-emergence safeners (soil-acting safeners), for example
   “dichlormid” (N,N-diallyl-2,2-dichloroacetamide) (S3-1),
   “R-29148” (3-dichloroacetyl-2,2,5-trimethyl-1,3-oxazolidine) from Stauffer (S3-2),
   “R-28725” (3-dichloroacetyl-2,2-dimethyl-1,3-oxazolidine) from Stauffer (S3-3),
   “benoxacor” (4-dichloroacetyl-3,4-dihydro-3-methyl-2H-1,4-benzoxazine) (S3-4),
   “PPG-1292” (N-allyl-N-[(1,3-dioxolan-2-yl)methyl]dichloroacetamide) from PPG Industries (S3-5),
   “DKA-24” (N-allyl-N-[(allylaminocarbonyl)methyl]dichloroacetamide) from Sagro-Chem (S3-6),
   “AD-67” or “MON 4660” (3-dichloroacetyl-1-oxa-3-azaspiro[4.5]decane) from Nitrokemia or Monsanto (S3-7),
   “TI-35” (1-dichloroacetylazepane) from TRI-Chemical RT (S3-8),
   “Diclonon” (Dicyclonon) or “BAS 145138” or “LAB 145138” (S3-9)
   ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyloxazolidine) (S3-10); and the (R) isomer thereof (S3-11).
S4)N-acylsulfonamides of the formula (S4) and salts thereof,

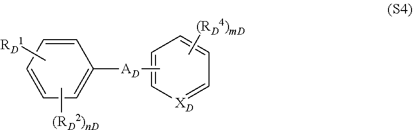

in which the symbols and indices are defined as follows:
A_{D} is SO₂—NR_{D}³-CO or CO—NR_{D}³—SO₂
X_{D} is CH or N;
R_{D}¹ is CO—NR_{D}⁵R_{D}⁶ or NHCO—R_{D}⁷;
R_{D}² is halogen, (C₁-C₄)-haloalkyl, (C₁-C₄)-haloalkoxy, nitro, (C₁-C₄)-alkyl, (C₁-C₄)-alkoxy, (C₁-C₄)-alkylsulfonyl, (C₁-C₄)-alkoxycarbonyl or (C₁-C₄)-alkylcarbonyl;
R_{D}³ is hydrogen, (C₁-C₄)-alkyl, (C₂-C₄)-alkenyl or (C₂-C₄)-alkynyl;
R_{D}⁴ is halogen, nitro, (C₁-C₄)-alkyl, (C₁-C₄)-haloalkyl, (C₁-C₄)-haloalkoxy, (C₃-C₆)-cycloalkyl, phenyl, (C₁-C₄)-alkoxy, cyano, (C₁-C₄)-alkylthio, (C₁-C₄)-alkylsulfinyl, (C₁-C₄)-alkylsulfonyl, (C₁-C₄)-alkoxycarbonyl or (C₁-C₄)-alkylcarbonyl;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl, phenyl or 3- to 6-membered heterocyclyl containing v_{D} heteroatoms from the group consisting of nitrogen, oxygen and sulfur, where the seven last-mentioned radicals are substituted by v_{D} substituents from the group consisting of halogen, (C₁-C₄)-alkoxy, (C₁-C₆)-haloalkoxy, (C₁-C₂)-alkylsulfinyl, (C₁-C₂)-alkylsulfonyl, (C₃-C₆)-cycloalkyl, (C₁-C₄)-alkoxycarbonyl, (C₁-C₄)-alkylcarbonyl and phenyl and, in the case of cyclic radicals, also (C₁-C₄)-alkyl and (C₁-C₄)-haloalkyl;
R_{D}⁶ is hydrogen, (C₁-C₆)-alkyl, (C₂-C₆)-alkenyl or (C₂-C₆)-alkynyl, where the three last-mentioned radicals are substituted by v_{D} radicals from the group consisting of halogen, hydroxy, (C₁-C₄)-alkyl, (C₁-C₄)-alkoxy and (C₁-C₄)-alkylthio, or
R_{D}⁵ and R_{D}⁶ together with the nitrogen atom carrying them form a pyrrolidinyl or piperidinyl radical;
R_{D}⁷ is hydrogen, (C₁-C₄)-alkylamino, di-(C₁-C₄)-alkylamino, (C₁-C₆)-alkyl, (C₃-C₆)-cycloalkyl, where the 2 last-mentioned radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
n_{D} is 0, 1 or 2;
m_{D} is 1 or 2;
v_{D} is 0, 1, 2 or 3;
   among these, preference is given to compounds of the N-acylsulfonamide type, for example of the formula (S4^{a}) below, which are known, for example, from WO-A-97/45016

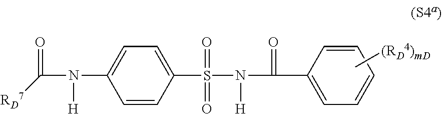

in which
R_{D}⁷ is (C₁-C₆)-alkyl, (C₃-C₆)-cycloalkyl, where the 2 last-mentioned radicals are substituted by v_{D} substituents from the group consisting of halogen, (C₁-C₄)-alkoxy, (C₁-C₆)-haloalkoxy and (C₁-C₄)-alkylthio and, in the case of cyclic radicals, also (C₁-C₄)-alkyl and (C₁-C₄)-haloalkyl;
R_{D}⁴ is halogen, (C₁-C₄)-alkyl, (C₁-C₄)-alkoxy, CF₃;
m_{D} is 1 or 2;
v_{D} is 0, 1, 2 or 3;
and
   acylsulfamoylbenzamides, for example of the formula (S4^{b}) below, which are known, for example, from WO-A-99/16744,

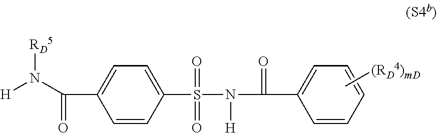

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-Cl-2-OMe (S4-4) and
R_{D}⁵=isopropyl and (R_{D}⁴)=2-OMe (S4-5)
and
   compounds of the N-acylsulfamoylphenylurea type, of the formula (S4), which are known, for example, from EP-A-365484,

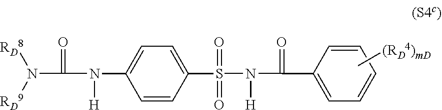

in which
R_{D}⁸ and R_{D}⁹ independently of one another are hydrogen, (C₁-C₆)-alkyl, (C₃-C₅)-cycloalkyl, (C₃-C₆)-alkenyl, (C₃-C₆)-alkynyl,
R_{D}⁴ is halogen, (C₁-C₄)-alkyl, (C₁-C₄)-alkoxy, CF₃
m_{D} is I or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea,
and
   N-phenylsulfonylterephthalamides of the formula (S4^{d}), which are known, for example, from CN 101838227,

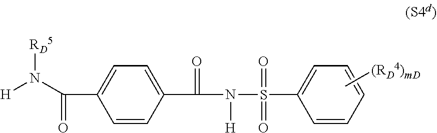

for example those in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C2-C₆)-alkynyl, (C₅-C₆)-cycloalkenyl.
S5) Active compounds from the class of the hydroxyaromatics and the aromatic-aliphatic carboxylic acid derivatives (S5), for example
   ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active compounds from the class of the 1,2-dihydroquinoxalin-2-ones (S6), for example 1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.

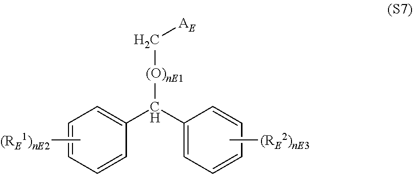

S7) Compounds of the formula (S7), as described in WO-A-1998/38856, in which the symbols and indices are defined as follows:
R_{E}¹, R_{E}² are each independently of one another halogen, (C₁-C₄)-alkyl, (C₁-C₄)-alkoxy, (C₁-C₄)-haloalkyl, (C₁-C₄)-alkylamino, di-(C₁-C₄)-alkylamino, nitro;
A_{E} is COOR_{E}³ or COSR_{E}⁴,
R_{E}³, R_{E}⁴ are each independently of one another hydrogen, (C₁-C₄)-alkyl, (C₂-C₅)-alkenyl, (C₂-C₄)-alkynyl, cyanoalkyl, (C₁-C₄)-haloalkyl, phenyl, nitrophenyl, benzyl, halobenzyl, pyridinylalkyl and alkylammonium,
n_{E}¹ is 0 or 1,
n_{E}², n_{E}³ are each independently 0, 1 or 2, preferably:
diphenylmethoxyacetic acid,
ethyl diphenylmethoxyacetate,
methyl diphenylmethoxyacetate (CAS reg. no. 41858-19-9) (S7-1).
S8) Compounds of the formula (S8), as described in WO-A-98/27049,

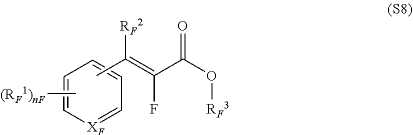

in which
X_{F} is CH or N,
n_{F} in the case that X═N is an integer from 0 to 4 and in the case that X_{F}═CH is an integer from 0 to 5,
R_{F}¹ is halogen, (C₁-C₄)-alkyl, (C₁-C₄)-haloalkyl, (C₁-C₄)-alkoxy, (C₁-C₄)-haloalkoxy, nitro, (C₁-C₄)-alkylthio, (C₁-C₄)-alkylsulfonyl, (C₁-C₄)-alkoxycarbonyl, optionally substituted phenyl, optionally substituted phenoxy,
R_{F}² is hydrogen or (C₁-C₄)-alkyl,
R_{F}³ is hydrogen, (C₁-C₆)-alkyl, (C₂-C₄)-alkenyl, (C₂-C₄)-alkynyl or aryl, where each of the carbon-containing radicals mentioned above is unsubstituted or substituted by one or more, preferably up to three, identical or different radicals from the group consisting of halogen and alkoxy, or salts thereof,
preferably compounds in which
X_{F} is CH,
n_{F} is an integer from 0 to 2,
R_{F}¹ is halogen, (C₁-C₄)-alkyl, (C₁-C₄)-haloalkyl, (C₁-C₄)-alkoxy, (C₁-C₄)-haloalkoxy,
R_{F}² is hydrogen or (C₁-C₄)-alkyl,
R_{F}³ is hydrogen, (C₁-C₆)-alkyl, (C₂-C₄)-alkenyl, (C₂-C₄)-alkynyl, or aryl, where each of the aforementioned carbon-containing radicals is unsubstituted or substituted by one or more, preferably up to three identical or different radicals from the group consisting of halogen and alkoxy,
or salts thereof.
S9) active compounds from the class of the 3-(5-tetrazolylcarbonyl)-2-quinolones (S9), for example 1,2-dihydro-4-hydroxy-1-ethyl-3-(5-tetrazolylcarbonyl)-2-quinolone (CAS reg. no. 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formula (S10^{a}) or (S10^{b})
   as described in WO-A-2007/023719 and WO-A-2007/023764

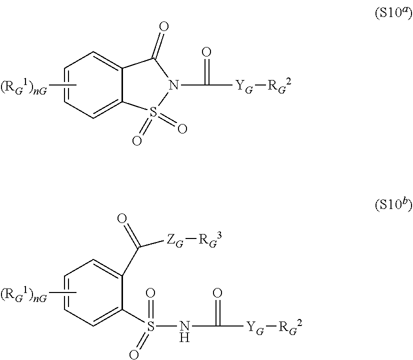

in which
R_{G}¹ is halogen, (C₁-C₄)-alkyl, methoxy, nitro, cyano, CF₃, OCF₃,
Y_{G}, Z_{G} are each independently O or S,
n_{G} is an integer from 0 to 4,
R_{G}² is (Ci-Cl₆)-alkyl, (C₂-C₆)-alkenyl, (C₃-C₆)-cycloalkyl, aryl; benzyl, halobenzyl,
R_{G}³ is hydrogen or (C₁-C₆)-alkyl.
S11) Active compounds of the oxyimino compound type (S11), which are known as seed-dressing agents, for example
   “oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) active compounds from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage,
   “CL 304415” (CAS Reg. No. 31541-57-8)
   (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5)
   (2-propenyl 1-oxa-4-azaspiro[4.5]decane-4-carbodithioate) (S13-6) from Nitrokemia,
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active compounds which, in addition to herbicidal action against harmful plants, also have safener action on crop plants such as rice, for example
   “dimepiperate” or “MY 93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as safener for rice against imazosulfuron herbicide damage,
   “cumyluron”=“JC 940” (3-(2-chlorophenylmethyl)-1-(1-methyl-1-phenylethyl)urea, see JP-A-60087254), which is known as safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

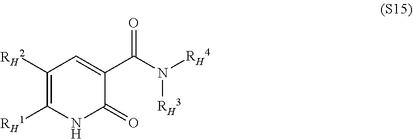

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ are each independently hydrogen, (C₁-C₁₆)-alkyl, (C₂-C₁₆)-alkenyl or (C₂-C₁₆)-alkynyl,
   where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C4-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
   where each of the 4 last-mentioned radicals is unsubstituted or substituted by one or more radicals from the group consisting of halogen, hydroxyl, cyano, (C₁-C₄)-alkyl, (C1-C4)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C4)-haloalkyl, (C₁-C₄)-alkoxy, (C₁-C₄)-haloalkoxy and (C₁-C₄)-alkylthio.
S16) Active compounds which are used primarily as herbicides but also have safener action on crop plants, for example
(2,4-dichlorophenoxy)acetic acid (2,4-D),
(4-chlorophenoxy)acetic acid,
(R,S)-2-(4-chloro-o-tolyloxy)propionic acid (mecoprop),
4-(2,4-dichlorophenoxy)butyric acid (2,4-DB),
(4-chloro-o-tolyloxy)acetic acid (MCPA),
4-(4-chloro-o-tolyloxy)butyric acid,
4-(4-chlorophenoxy)butyric acid,
3,6-dichloro-2-methoxybenzoic acid (dicamba),
1-(ethoxycarbonyl)ethyl 3,6-dichloro-2-methoxybenzoate (lactidichlor-ethyl).

Preferred safeners are: cloquintocet-mexyl, cyprosulfamide, fenchlorazole-ethyl, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

Wettable powders are preparations which can be dispersed uniformly in water and, in addition to the active compound, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the herbicidally active compounds are finely ground, for example in customary apparatuses such as hammer mills, blower mills and air-jet mills, and simultaneously or subsequently mixed with the formulation auxiliaries.

Emulsifiable concentrates are produced by dissolving the active compound in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonates such as calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active compound with finely distributed solids, for example talc, natural clays, such as kaolin, bentonite and pyrophyllite, or diatomaceous earth.

Suspension concentrates may be water- or oil-based. They may be prepared, for example, by wet-grinding by means of commercial bead mills and optional addition of surfactants as have, for example, already been listed above for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active compound onto adsorptive granular inert material or by applying active compound concentrates to the surface of carriers, such as sand, kaolinites or granular inert material, by means of adhesives, for example polyvinyl alcohol, sodium polyacrylate or else mineral oils. Suitable active compounds can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan, fluidized-bed, extruder and spray granules, see e.g. processes in “Spray-Drying Handbook” 3rd Ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1 to 99% by weight, especially 0.1 to 95% by weight, of compounds of the invention.

In wettable powders, the active compound concentration is, for example, about 10 to 90% by weight, the remainder to 100% by weight consisting of customary formulation constituents. In emulsifiable concentrates, the active compound concentration may be about 1% to 90% and preferably 5% to 80% by weight. Formulations in the form of dusts comprise 1% to 30% by weight of active compound, preferably usually 5% to 20% by weight of active compound; sprayable solutions contain about 0.05% to 80% by weight, preferably 2% to 50% by weight of active compound. In the case of water-dispersible granules, the active compound content depends partially on whether the active compound is in liquid or solid form and on which granulation auxiliaries, fillers, etc., are used. In the water-dispersible granules, the content of active compound is, for example, between 1% and 95% by weight, preferably between 10% and 80% by weight.

In addition, the active compound formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tankmix. Active compounds which can be employed in combination with the compounds according to the invention in mixed formulations or in the tank mix are, for example, known active compounds which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoen desaturase, photosystem I, photosystem II, protoporphyrinogen oxidase, as are described in, for example, Weed Research 26 (1986) 441-445 or “The Pesticide Manual”, 15th edition, The British Crop Protection Council and the Royal Soc. of Chemistry, 2009 and the literature cited therein. Known herbicides or plant growth regulators which can be combined with the compounds according to the invention are, for example, the following active compounds (the compounds are either designated by the common name according to the International Organization for Standardization (ISO) or by the chemical name, or by the code number) and always comprise all use forms such as acids, salts, esters and isomers such as stereoisomers and optical isomers. These include, by way of example, one use form and in some cases also a plurality of use forms:

acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-b-(4-chloro-2-fluoro-3-Methylphenyl)-5-fluoropyridine-2-carboxylic acid, aminocyclopyrachlor, aminocyclopyrachlor-potassium, aminocyclopyrachlor-methyl, aminopyralid, amitrole, ammonium sulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat dibromide, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and -sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinate, monolinuron, monosulfuron, monosulfuron ester, MT-5950, i.e. N-[3-chloro-4-(1-methylethyl)phenyl]-2-methylpentanamide, NGGC-01 1, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trichloroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vemolate, XDE-848, ZJ-0862, i.e. 3,4-dichloro-N-{2-[(4,6-dimethoxypyrimidin-2-yl)oxy]benzyl}aniline, and the compounds below:

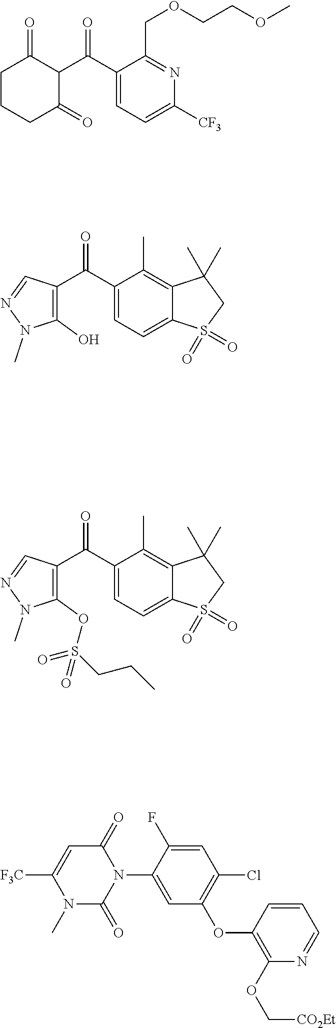 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl]

Examples of plant growth regulators as possible mixing partners are:

acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenoxide mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) varies with the external conditions, including, inter alia, temperature, humidity and the type of herbicide used. It can vary within wide limits, for example between 0.001 and 1.0 kg/ha or more of active substance, but it is preferably between 0.005 and 750 g/ha.

The examples which follow illustrate the invention in detail.

### A. Chemical Examples

1. Preparation of 3-(trifluoromethyl)benzyl 4-amino-6-chloro-(1-benzothiophen-5-yl)-3-chloropyridine-2-carboxylate (Example No. I-21)

In succession, 0.075 ml of triethylamine and 0.094 g (0.39 mmol) of 3-trifluorobenzyl bromide are added to a solution of 0.08 g (0.26 mmol) of 4-amino-6-(1-benzothiophen-5-yl)-3-chloropyridine-2-carboxylic acid in 10 ml of THF, and this reaction mixture is stirred at room temperature for 12 hours. For work-up, the mixture is concentrated to dryness and the resulting crude mixture is purified by column chromatography on silica gel (heptane/ethyl acetate 2/8). This gives 0.1 g (82%) of product as a colorless oil. 1H NMR, CDCl3; 8.40 (s, 1H, benzothiophene), 7.90 (m, 2H, benzothiophene), 7.70 (s, 1H, phenyl), 7.70, 7.60 (2d, 2H, phenyl), 7.50 (t, 1H, phenyl), 7.49, 7.40 (2d, 2H, benzothiophene), 7.20 (s, 1H, pyridine), 5.50 (s, 2H, CH2-phenyl), 4.70 (bs, 2H, NH2).

2. Preparation of 4-fluorobenzyl 4-amino-6-(1-benzothiophen-6-yl)-3-chloropyridine-2-carboxylate (Example No. I-07)

In succession, 0.09 ml of triethylamine and 0.093 g (0.49 mmol) of 4-fluorobenzyl bromide are added to a solution of 0.1 g (0.33 mmol) of 4-amino-6-(1-benzothiophen-6-yl)-3-chloropyridine-2-carboxylic acid in 10 ml of THF, and this reaction mixture is stirred at room temperature for 12 hours. For work-up, the mixture is concentrated to dryness and the resulting crude mixture is purified by column chromatography on silica gel (heptane/ethyl acetate 2/8). This gives 0.09 g (66%) of product as a colorless oil. 1H-NMR, CDCl3; 8.50 (s, benzothiophene), 7.85 (m, 2H, benzothiophene), 7.50 (m, 3H, benzyl, benzothiophene), 7.35 (d, 1H, benzothiophene), 7.25 (s, 1H, pyridine), 7.08 (m, 2H, benzyl), 5.95 (s, 2H, OCH2), 4.70 (bs, 2H, NH2).

3. Preparation of 3-fluorobenzyl 4-amino-3-chloro-5-fluoro-6-(7-fluoro-1H-indol-6-yl)pyridine-2-carboxylate (Example No. 1-84)

In succession, 0.09 ml of triethylamine and 0.088 g (0.47 mmol) of 3-fluorobenzyl bromide are added to a solution of 0.1 g (0.31 mmol) of 4-amino-3-chloro-5-fluoro-6-(7-fluoro-1H-indol-6-yl)pyridine-2-carboxylic acid in 11 ml of THF, and this reaction mixture is briefly heated to 70° C. and stirred at room temperature for 12 hours. For work-up, the mixture is concentrated to dryness and the resulting crude mixture is purified by column chromatography on silica gel (heptane/ethyl acetate 2/8). This gives 0.1 g (79%) of product as a colorless oil. 1H-NMR, CDCl3; 8.45 (bs, NH, indole), 7.50 (d, 1H, indole), 7.30 (m, 5H, indole, benzyl), 7.20 (m, 1H, benzyl), 6.60 (m, 1H, indole), 5.40 (2H, CH2-benzyl), 4.90 (bs, 2H, NH2).

NMR Data of Selected Examples

NMR Peak List Method

The 1H-NMR data of selected examples are noted in the form of 1H-NMR peak lists. For each signal peak, first the δ value in ppm and then the signal intensity in round brackets is listed. The δ value—signal intensity number pairs for different signal peaks are listed with separation from one another by semicolons.

The peak list for one example therefore takes the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists.

The lists of the 1H NMR peaks are similar to the conventional 1H NMR printouts and thus usually contain all peaks listed in a conventional NMR interpretation.

In addition, like conventional 1H NMR printouts, they may show solvent signals, signals of stereoisomers of the target compounds, which likewise form part of the subject-matter of the invention, and/or peaks of impurities.

In the reporting of compound signals in the delta range of solvents and/or water, our lists of 1H NMR peaks show the usual solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of 1H NMR peak lists can be found in the Research Disclosure Database Number 564025.

Example I-01: ¹H-NMR(400.0 MHz, CDCl3): δ =
8.498(6.6); 8.496(7.0); 8.494(6.5); 7.896(3.2); 7.892(3.0); 7.875(8.2); 7.871(8.2); 7.849(9.0); 7.848(9.0);
7.828(3.4); 7.604(1.6); 7.600(1.7); 7.586(3.3); 7.581(3.3); 7.567(1.7); 7.563(1.8); 7.501(12.2); 7.488(14.5);
7.365(1.1); 7.360(1.1); 7.351(1.4); 7.345(8.8); 7.343(8.2); 7.332(7.5); 7.330(7.1); 7.326(2.4); 7.321(1.5);
7.312(1.4); 7.308(1.4); 7.264(0.6); 7.263(0.7); 7.262(0.8); 7.261(1.0); 7.256(63.5); 7.252(0.6); 7.192(2.9);
7.189(3.2); 7.173(4.9); 7.170(5.3); 7.159(19.8); 7.154(2.5); 7.151(2.4); 7.122(2.7); 7.119(2.4); 7.101(2.3);
7.097(3.6); 7.094(2.5); 7.076(2.1); 7.073(2.0); 5.543(16.0); 5.294(8.7); 4.779(6.3); 1.565(1.6); 0.008(0.8);
0.000(28.9); −0.008(0.8)
Example I-02: ¹H-NMR(400.0 MHz, CDCl3): δ =
8.110(2.9); 8.107(4.2); 8.106(5.6); 8.104(4.2); 8.102(2.8); 7.833(4.3); 7.829(4.1); 7.812(5.2); 7.809(5.2);
7.678(8.7); 7.672(8.7); 7.642(5.3); 7.641(5.3); 7.622(4.4); 7.621(4.2); 7.384(0.8); 7.382(0.7); 7.369(1.2);
7.366(1.1); 7.364(2.1); 7.349(2.5); 7.343(1.6); 7.329(2.0); 7.280(3.1); 7.278(2.4); 7.262(4.9); 7.260(5.3);
7.258(53.5); 7.254(1.7); 7.252(1.1); 7.240(1.3); 7.239(1.4); 7.238(1.1); 7.234(1.7); 7.230(1.2); 7.157(18.4);
7.058(1.0); 7.056(1.0); 7.052(0.9); 7.049(0.9); 7.037(1.5); 7.034(1.7); 7.030(1.4); 7.028(1.5); 7.016(0.8);
7.013(0.8); 7.010(0.8); 7.007(0.8); 6.788(5.5); 6.785(5.7); 6.782(5.6); 6.780(5.3); 5.452(16.0); 5.296(0.8);
4.788(4.1); 1.549(10.2); 0.008(0.7); 0.000(22.6); −0.008(0.6)
Example I-03: ¹H-NMR(400.0 MHz, CDCl3): δ =
8.111(1.4); 7.880(0.6); 7.876(1.1); 7.872(0.6); 7.860(0.7); 7.856(1.3); 7.852(0.7); 7.705(0.6); 7.700(2.9);
7.695(2.9); 7.692(0.8); 7.673(1.8); 7.652(1.4); 7.442(2.4); 7.437(1.1); 7.425(0.8); 7.420(2.6); 7.308(0.7);
7.286(0.7); 7.258(49.5); 6.925(3.0); 6.920(1.0); 6.908(1.5); 6.903(3.0); 6.886(0.8); 6.858(0.5); 6.806(1.6);
6.803(1.6); 6.800(1.6); 6.798(1.5); 6.661(0.6); 5.384(6.1); 4.852(1.4); 4.632(0.6); 4.617(0.6); 3.817(16.0);
3.812(4.7); 3.778(2.9); 1.536(13.4); 0.008(0.7); 0.000(21.0); −0.008(0.6)
Example I-04: ¹H-NMR(400.0 MHz, CDCl3): δ =
8.196(0.9); 8.115(4.4); 7.969(0.7); 7.949(0.9); 7.882(2.0); 7.878(3.5); 7.874(2.0); 7.861(2.4); 7.857(4.3);
7.854(2.3); 7.709(2.3); 7.706(8.4); 7.700(9.6); 7.697(2.6); 7.680(6.4); 7.659(4.6); 7.604(1.0); 7.585(1.1);
7.517(1.0); 7.499(3.7); 7.494(1.6); 7.486(3.9); 7.477(4.2); 7.469(1.7); 7.464(4.1); 7.409(1.2); 7.356(1.0);
7.343(1.1); 7.334(1.3); 7.320(1.3); 7.309(0.7); 7.258(157.8); 7.209(1.1); 7.155(1.0); 7.151(1.0); 7.135(1.0);
7.132(1.0); 7.105(0.7); 7.097(5.0); 7.092(1.7); 7.081(1.8); 7.076(9.2); 7.070(1.9); 7.067(2.0); 7.059(1.7);
7.054(4.5); 7.045(2.8); 7.039(0.6); 7.023(1.2); 6.994(0.9); 6.929(0.7); 6.915(0.8); 6.907(1.0); 6.893(1.1);
6.828(1.2); 6.826(1.2); 6.822(1.8); 6.816(1.6); 6.814(1.8); 6.811(5.4); 6.808(5.6); 6.806(5.9); 6.803(5.2);
6.785(1.9); 6.763(0.8); 5.408(16.0); 5.004(1.2); 4.872(4.2); 4.675(1.8); 4.661(1.8); 4.380(1.0); 1.623(0.6);
1.609(1.2); 1.594(0.6); 1.533(31.0); 1.333(0.6); 1.284(0.8); 1.256(0.7); 0.008(2.3); 0.000(67.5); −0.008(2.3)
Example I-05: ¹H-NMR(400.0 MHz, CDCl3): δ =
8.166(5.1); 8.162(5.2); 7.853(3.2); 7.848(3.1); 7.831(3.7); 7.827(3.6); 7.647(6.9); 7.641(6.9); 7.542(4.5);
7.540(3.1); 7.522(2.8); 7.520(4.0); 7.518(2.8); 7.503(3.8); 7.498(1.6); 7.490(4.0); 7.481(4.4); 7.473(1.7);
7.468(4.1); 7.258(66.9); 7.122(16.1); 7.101(0.6); 7.093(5.2); 7.088(1.6); 7.077(1.9); 7.072(9.7); 7.066(1.7);
7.055(1.8); 7.050(4.6); 6.810(5.0); 6.807(5.1); 6.804(5.0); 6.802(4.8); 5.421(16.0); 5.296(1.4); 4.763(4.4);
1.551(12.6); 0.008(0.8); 0.000(25.2); −0.008(0.7)
Example I-06: ¹H-NMR(400.0 MHz, CDCl3): δ =
8.192(7.6); 8.189(7.6); 7.861(5.0); 7.857(4.8); 7.840(5.7); 7.835(5.4); 7.653(10.7); 7.648(10.5); 7.555(5.0);
7.553(6.9); 7.551(4.7); 7.534(4.3); 7.532(6.0); 7.530(4.5); 7.527(1.8); 7.518(0.6); 7.510(1.7); 7.505(1.8);
7.501(1.8); 7.488(1.8); 7.484(1.8); 7.479(1.7); 7.463(1.5); 7.268(0.5); 7.267(0.6); 7.266(0.7); 7.264(1.1);
7.264(1.3); 7.259(87.5); 7.255(0.8); 7.254(0.6); 7.209(0.6); 7.164(0.5); 7.151(27.6); 7.005(2.2); 6.995(0.7);
6.989(2.3); 6.981(2.6); 6.964(2.6); 6.957(2.2); 6.941(2.1); 6.823(8.0); 6.821(8.1); 6.818(8.0); 6.815(7.7);
5.457(16.0); 5.297(11.7); 4.795(6.5); 1.552(9.8); 1.255(0.6); 0.008(1.2); 0.000(37.8); −0.008(1.1)
Example I-08: ¹H-NMR(400.0 MHz, CDCl3): δ =
8.495(2.5); 8.493(2.7); 8.491(2.6); 7.893(1.0); 7.890(1.0); 7.872(3.2); 7.868(3.3); 7.854(3.6); 7.852(3.5);
7.833(1.1); 7.832(1.0); 7.507(4.8); 7.494(5.6); 7.350(2.7); 7.348(2.7); 7.337(2.3); 7.335(2.2); 7.258(26.6);
7.244(0.7); 7.230(0.7); 7.225(0.8); 7.224(0.8); 7.223(0.8); 7.211(0.8); 7.208(1.0); 7.204(0.8); 7.189(0.8);
7.171(7.6); 6.946(1.0); 6.940(1.0); 6.924(1.0); 6.919(1.6); 6.915(1.1); 6.898(0.9); 6.893(0.9); 5.477(4.9);
5.476(5.1); 5.474(4.9); 4.791(2.5); 4.017(8.5); 4.014(16.0); 4.012(8.4); 1.559(6.1); 0.000(11.7)
Example I-09: ¹H-NMR(400.0 MHz, CDCl3): δ =
8.411(4.8); 8.409(4.6); 7.931(1.7); 7.910(7.2); 7.899(5.0); 7.895(4.7); 7.878(1.2); 7.874(1.2); 7.474(4.9);
7.460(7.0); 7.398(5.4); 7.384(3.8); 7.379(1.2); 7.365(1.2); 7.360(2.4); 7.345(2.7); 7.340(1.8); 7.327(1.0);
7.324(1.6); 7.280(2.0); 7.275(3.9); 7.273(4.2); 7.257(45.7); 7.184(14.8); 7.059(0.9); 7.057(1.0); 7.053(0.9);
7.050(0.9); 7.036(1.7); 7.031(1.6); 7.017(0.9); 7.013(1.0); 7.008(0.8); 6.858(0.7); 5.457(16.0); 5.381(0.8);
5.294(12.8); 4.797(4.4); 1.561(1.2); 0.008(0.5); 0.000(15.8)
Example I-10: ¹H-NMR(400.0 MHz, CDCl3): δ =
8.509(2.8); 8.507(4.8); 8.505(5.3); 8.503(5.0); 8.501(2.7); 7.908(2.4); 7.904(2.3); 7.887(6.1); 7.883(6.1);
7.861(6.2); 7.860(6.3); 7.840(2.4); 7.839(2.3); 7.508(8.1); 7.494(9.4); 7.384(1.3); 7.369(1.2); 7.363(3.1);
7.357(0.5); 7.351(5.6); 7.349(7.2); 7.343(2.4); 7.338(4.1); 7.336(4.1); 7.329(2.0); 7.278(3.1); 7.276(2.6);
7.272(1.8); 7.268(2.0); 7.266(2.1); 7.262(3.4); 7.256(37.5); 7.248(1.4); 7.248(1.5); 7.242(1.7); 7.238(1.1);
7.179(16.4); 7.061(0.9); 7.058(1.0); 7.055(0.9); 7.052(0.9); 7.040(1.6); 7.038(1.6); 7.033(1.5); 7.032(1.4);
7.019(0.8); 7.016(0.8); 7.013(0.8); 7.010(0.7); 5.458(16.0); 5.294(3.1); 4.797(4.4); 1.562(2.6); 0.008(0.5);
0.000(16.1)
Example I-11: ¹H-NMR(400.0 MHz, CDCl3): δ =
8.203(1.0); 8.200(1.0); 7.877(0.6); 7.872(0.6); 7.855(0.8); 7.851(0.7); 7.829(0.7); 7.655(1.4); 7.650(1.4);
7.558(0.7); 7.556(0.9); 7.554(0.6); 7.536(0.9); 7.535(1.0); 7.533(0.6); 7.518(0.9); 7.259(49.0); 7.163(4.0);
6.826(1.0); 6.823(1.0); 6.820(1.1); 6.818(1.0); 5.513(3.1); 4.786(0.8); 1.538(16.0); 0.008(0.6); 0.000(19.9);
−0.008(0.6)
Example I-12: ¹H-NMR(400.0 MHz, CDCl3): δ =
8.497(4.2); 8.495(7.6); 8.493(8.2); 8.491(7.8); 8.489(4.0); 7.896(3.2); 7.892(3.0); 7.875(10.3); 7.871(10.4);
7.857(10.2); 7.856(9.9); 7.836(3.1); 7.834(2.8); 7.597(1.6); 7.581(1.8); 7.576(3.4); 7.560(3.4); 7.555(1.9);
7.538(1.8); 7.517(0.6); 7.509(12.5); 7.495(14.7); 7.352(7.5); 7.350(7.3); 7.338(6.3); 7.336(6.2); 7.258(76.0);
7.172(27.0); 6.936(1.4); 6.933(1.3); 6.930(1.6); 6.927(1.6); 6.915(2.1); 6.913(2.2); 6.908(2.6); 6.906(2.8);
6.894(1.3); 6.892(1.3); 6.887(3.6); 6.881(1.8); 6.864(2.8); 6.862(2.8); 6.858(2.1); 6.856(2.2); 6.840(2.4);
6.834(1.9); 5.486(16.0); 5.296(6.0); 4.785(6.6); 1.553(8.3); 0.008(0.9); 0.000(32.3); −0.008(1.0)
Example I-13: ¹H-NMR(400.0 MHz, CDCl3): δ =
8.166(7.9); 8.163(8.0); 7.852(5.2); 7.847(5.0); 7.830(5.9); 7.826(5.6); 7.651(11.0); 7.645(10.9); 7.548(5.2);
7.546(7.2); 7.544(4.8); 7.526(4.5); 7.524(6.2); 7.522(4.2); 7.518(0.6); 7.341(1.1); 7.335(1.1); 7.327(1.2);
7.321(2.0); 7.319(1.9); 7.313(1.4); 7.309(1.2); 7.303(1.8); 7.301(2.0); 7.295(1.2); 7.287(1.2); 7.281(1.2);
7.269(0.6); 7.259(86.0); 7.255(0.6); 7.209(0.6); 7.135(29.3); 7.028(1.5); 7.023(1.5); 7.011(1.6); 7.006(3.2);
6.999(1.9); 6.995(0.7); 6.988(1.9); 6.982(2.9); 6.977(1.3); 6.965(1.2); 6.960(1.2); 6.814(8.6); 6.811(8.5);
6.808(8.4); 6.806(8.0); 5.485(14.9); 5.484(16.0); 5.482(14.5); 5.297(2.7); 4.783(6.8); 1.554(9.9); 0.008(1.2);
0.000(38.3); −0.008(1.0)
Example I-14: ¹H-NMR(400.0 MHz, CDCl3): δ =
8.500(2.6); 8.498(4.7); 8.496(5.0); 8.494(4.8); 8.492(2.4); 7.900(2.0); 7.896(1.9); 7.879(6.4); 7.876(6.5);
7.868(0.7); 7.862(6.2); 7.860(6.1); 7.840(1.9); 7.839(1.7); 7.516(0.5); 7.511(8.2); 7.498(9.7); 7.467(0.7);
7.462(5.4); 7.460(4.5); 7.456(1.8); 7.445(2.4); 7.440(8.4); 7.434(1.1); 7.382(1.6); 7.376(10.9); 7.371(2.9);
7.360(2.5); 7.355(10.8); 7.349(0.8); 7.341(4.1); 7.339(3.9); 7.265(0.6); 7.264(0.7); 7.263(0.8); 7.257(56.6);
7.253(0.6); 7.178(16.0); 5.428(16.0); 5.296(4.1); 4.786(4.0); 1.548(1.0); 0.070(2.3); 0.008(0.7); 0.000(26.3);
−0.008(0.8)
Example I-15: ¹H-NMR(400.0 MHz, CDCl3): δ =
8.197(0.9); 8.118(3.9); 7.971(0.6); 7.951(0.8); 7.885(1.7); 7.881(3.1); 7.877(1.7); 7.865(2.1); 7.861(3.8);
7.857(2.0); 7.711(3.3); 7.708(7.5); 7.706(4.1); 7.703(8.4); 7.682(5.7); 7.663(4.0); 7.597(0.9); 7.576(1.0);
7.517(0.9); 7.450(4.7); 7.445(1.9); 7.434(2.4); 7.428(8.2); 7.407(1.2); 7.379(1.6); 7.374(10.5); 7.369(3.0);
7.358(2.3); 7.352(5.7); 7.309(1.4); 7.258(151.8); 7.210(1.0); 7.148(0.8); 7.144(0.9); 7.128(0.8); 7.124(0.7);
7.079(1.5); 7.075(0.6); 7.063(0.6); 7.058(1.9); 6.994(0.8); 6.876(1.6); 6.855(1.4); 6.832(1.0); 6.830(1.1);
6.827(1.1); 6.823(1.2); 6.820(1.2); 6.818(1.3); 6.813(4.6); 6.811(4.5); 6.808(4.5); 6.805(4.4); 5.407(16.0);
5.297(0.9); 5.004(1.1); 4.879(3.7); 4.385(0.9); 1.534(10.0); 1.284(0.7); 1.255(0.7); 0.008(1.9); 0.000(64.5);
−0.008(2.3)
Example I-16: ¹H-NMR(400.0 MHz, CDCl3): δ =
8.396(6.0); 8.394(5.7); 8.392(5.4); 7.938(2.6); 7.917(9.1); 7.903(6.1); 7.899(5.8); 7.881(1.7); 7.877(1.7);
7.669(2.7); 7.653(2.6); 7.647(13.6); 7.636(11.5); 7.615(2.4); 7.482(6.3); 7.468(8.4); 7.395(6.3); 7.394(6.5);
7.381(4.7); 7.380(4.7); 7.257(58.5); 7.193(20.3); 6.875(1.0); 5.516(16.0); 5.444(0.8); 5.294(2.7); 4.802(5.7);
1.548(9.3); 1.257(0.5); 0.071(0.7); 0.008(0.8); 0.000(23.9); −0.008(0.7)
Example I-17: ¹H-NMR(400.0 MHz, CDCl3): δ =
8.086(3.1); 7.801(2.1); 7.797(2.0); 7.780(2.6); 7.777(2.5); 7.670(4.8); 7.664(4.8); 7.624(3.2); 7.604(2.6);
7.258(14.5); 7.242(0.7); 7.227(0.8); 7.223(0.9); 7.220(0.9); 7.208(1.0); 7.206(1.0); 7.201(0.8); 7.187(0.8);
7.132(7.8); 6.943(1.1); 6.938(1.1); 6.921(1.0); 6.917(1.7); 6.912(1.1); 6.895(1.0); 6.890(0.9); 6.780(3.0);
6.777(3.0); 6.774(3.0); 6.772(2.8); 5.470(5.7); 5.468(5.9); 5.467(5.5); 5.294(14.4); 4.792(2.9); 4.014(8.8);
4.012(16.0); 4.009(8.5); 0.000(6.2)
Example I-18: ¹H-NMR(400.0 MHz, CDCl3): δ =
8.385(7.0); 8.383(6.8); 7.925(3.2); 7.903(10.8); 7.888(7.0); 7.884(6.8); 7.867(2.0); 7.863(2.2); 7.592(1.6);
7.576(1.8); 7.571(3.4); 7.555(3.4); 7.550(1.9); 7.534(1.8); 7.474(7.7); 7.460(10.3); 7.388(7.6); 7.387(7.7);
7.375(5.6); 7.374(5.5); 7.257(75.6); 7.169(24.4); 6.929(1.3); 6.926(1.3); 6.923(1.6); 6.920(1.6); 6.908(2.2);
6.906(2.2); 6.901(2.8); 6.899(2.9); 6.887(1.5); 6.884(3.3); 6.878(2.9); 6.861(3.0); 6.859(3.0); 6.855(2.1);
6.852(2.3); 6.848(1.1); 6.836(2.5); 6.830(2.0); 5.484(16.0); 5.415(0.6); 5.295(8.2); 4.781(6.5); 1.550(16.9);
0.008(0.9); 0.000(28.4); −0.008(0.8)
Example I-19: ¹H-NMR(400.0 MHz, CDCl3): δ =
8.386(4.4); 8.383(4.2); 8.382(4.0); 7.930(1.3); 7.928(2.3); 7.926(1.3); 7.908(4.1); 7.907(7.2); 7.905(4.1);
7.890(4.4); 7.886(4.2); 7.869(1.3); 7.865(1.4); 7.476(4.8); 7.462(7.1); 7.457(5.6); 7.456(4.7); 7.452(2.0);
7.441(2.5); 7.435(8.7); 7.430(1.4); 7.391(4.8); 7.390(5.0); 7.377(4.8); 7.371(11.2); 7.366(3.1); 7.355(2.4);
7.350(6.6); 7.344(0.8); 7.263(0.5); 7.262(0.6); 7.262(0.8); 7.257(52.0); 7.173(15.7); 5.426(16.0); 5.294(7.4);
4.785(4.0); 1.556(2.1); 0.008(0.6); 0.000(19.6); −0.008(0.6)
Example I-20: ¹H-NMR(400.0 MHz, CDCl3): δ =
8.165(6.8); 8.164(7.1); 8.160(7.1); 8.159(6.6); 7.847(4.6); 7.842(4.4); 7.825(5.2); 7.820(5.0); 7.654(0.7);
7.647(0.8); 7.642(10.0); 7.637(9.9); 7.536(4.8); 7.534(6.2); 7.532(4.7); 7.518(1.0); 7.514(4.1); 7.512(5.5);
7.510(4.0); 7.269(0.6); 7.269(0.6); 7.268(0.7); 7.267(0.8); 7.266(0.9); 7.265(1.1); 7.265(1.4); 7.264(1.6);
7.263(2.1); 7.262(2.8); 7.259(115.9); 7.256(1.7); 7.255(1.1); 7.254(0.8); 7.253(0.6); 7.252(0.5); 7.209(0.8);
7.156(0.5); 7.114(30.2); 6.995(0.6); 6.807(7.8); 6.805(7.9); 6.802(7.8); 6.799(7.5); 6.750(0.6); 6.742(1.0);
6.734(5.3); 6.728(0.8); 6.716(6.7); 6.712(6.8); 6.700(0.9); 6.694(5.2); 6.691(2.3); 6.686(1.0); 5.489(16.0);
5.297(4.9); 4.755(6.0); 4.130(0.6); 4.112(0.6); 3.636(0.5); 3.476(0.8); 2.043(2.8); 1.551(9.0); 1.282(0.5);
1.276(1.0); 1.258(2.7); 1.240(1.0); 0.938(0.6); 0.920(1.4); 0.902(0.6); 0.008(1.4); 0.000(53.4); −0.008(1.4)
Example I-22: ¹H-NMR(400.0 MHz, CDCl3): δ =
8.373(1.6); 8.371(1.6); 7.913(0.7); 7.891(2.2); 7.874(1.6); 7.870(1.5); 7.849(0.5); 7.467(1.7); 7.454(2.3);
7.378(1.7); 7.364(1.3); 7.257(7.6); 7.214(0.6); 7.199(0.6); 7.195(0.5); 7.153(4.0); 6.936(0.6); 6.931(0.6);
6.914(0.6); 6.910(1.0); 6.905(0.6); 6.888(0.5); 6.883(0.5); 5.471(3.4); 5.293(16.0); 4.794(1.8); 4.009(4.9);
4.007(8.5); 4.004(5.0); 1.256(0.5); 0.000(3.2)
Example I-23: ¹H-NMR(400.0 MHz, CDCl3): δ =
8.105(1.8); 8.103(2.5); 8.101(3.3); 8.100(2.4); 8.097(1.7); 7.819(2.6); 7.815(2.4); 7.799(3.2); 7.795(3.1);
7.684(5.1); 7.678(5.0); 7.645(3.1); 7.644(3.1); 7.625(2.5); 7.624(2.4); 7.338(0.7); 7.337(0.7); 7.320(0.7);
7.318(0.7); 7.269(0.5); 7.267(0.7); 7.266(0.8); 7.259(74.4); 7.255(0.5); 7.209(0.5); 7.168(11.6); 7.041(0.5);
7.036(0.5); 7.024(0.6); 7.018(1.1); 7.012(0.7); 7.000(0.7); 6.995(1.5); 6.794(3.2); 6.791(3.4); 6.788(3.2);
6.786(3.2); 5.491(5.2); 5.489(5.7); 5.488(5.0); 5.298(0.6); 4.788(2.4); 1.538(16.0); 0.008(1.0); 0.000(32.3);
−0.008(0.9)
Example I-24: ¹H-NMR(400.0 MHz, CDCl3): δ =
8.164(7.2); 8.160(7.3); 7.850(4.5); 7.846(4.3); 7.829(5.1); 7.824(4.9); 7.646(9.8); 7.640(9.8); 7.586(1.7);
7.570(1.8); 7.565(3.5); 7.549(3.5); 7.544(2.3); 7.541(4.7); 7.539(6.5); 7.528(1.9); 7.517(5.8); 7.258(75.8);
7.120(21.7); 6.927(1.3); 6.924(1.3); 6.921(1.6); 6.918(1.7); 6.906(2.2); 6.904(2.2); 6.897(3.0); 6.885(1.4);
6.882(3.3); 6.876(2.8); 6.859(2.9); 6.857(2.9); 6.853(2.1); 6.850(2.3); 6.834(2.5); 6.828(2.0); 6.808(6.9);
6.805(7.0); 6.802(7.0); 6.800(6.7); 5.477(16.0); 5.295(2.5); 4.768(6.3); 1.555(18.5); 0.008(0.8); 0.000(27.3);
−0.008(0.8)
Example I-25: ¹H-NMR(400.0 MHz, CDCl3): δ =
8.487(4.4); 8.485(8.0); 8.484(8.6); 8.482(8.5); 8.480(4.4); 7.892(2.6); 7.888(2.3); 7.871(10.9); 7.868(11.3);
7.859(10.9); 7.858(10.9); 7.838(2.4); 7.837(2.2); 7.517(0.9); 7.513(13.7); 7.506(0.5); 7.499(15.9); 7.353(7.8);
7.351(7.8); 7.339(6.7); 7.337(6.6); 7.267(0.6); 7.266(0.6); 7.266(0.8); 7.265(0.9); 7.264(1.1); 7.263(1.3);
7.258(91.8); 7.255(2.8); 7.254(2.0); 7.253(1.6); 7.252(1.2); 7.252(1.0); 7.251(0.8); 7.250(0.7); 7.249(0.6);
7.248(0.5); 7.248(0.5); 7.208(0.7); 7.184(26.0); 5.542(9.0); 5.538(16.0); 5.534(9.1); 5.296(8.1); 4.799(6.8);
2.044(1.6); 1.550(12.3); 1.276(0.6); 1.258(1.5); 1.240(0.6); 0.008(1.3); 0.006(0.5); 0.005(0.6); 0.000(40.9);
−0.003(2.0); −0.004(0.8); −0.005(0.6); −0.008(1.2)
Example I-26: ¹H-NMR(400.0 MHz, CDCl3): δ =
8.110(1.7); 8.108(2.4); 8.106(3.3); 8.105(2.4); 8.102(1.6); 7.821(2.6); 7.818(2.5); 7.801(3.3); 7.797(3.1);
7.681(5.1); 7.676(5.1); 7.642(3.1); 7.641(3.1); 7.622(2.5); 7.620(2.5); 7.518(0.8); 7.512(2.2); 7.507(0.8);
7.499(2.3); 7.496(1.2); 7.490(2.5); 7.482(0.9); 7.477(2.4); 7.269(0.8); 7.268(0.9); 7.266(1.2); 7.259(114.2);
7.255(1.0); 7.254(0.7); 7.209(0.6); 7.158(11.5); 7.101(2.9); 7.096(0.9); 7.085(1.0); 7.079(5.4); 7.074(0.9);
7.063(0.9); 7.057(2.6); 6.995(0.6); 6.792(3.4); 6.789(3.3); 6.786(3.4); 6.784(3.2); 5.426(8.6); 4.769(2.3);
1.536(16.0); 0.008(1.4); 0.000(51.6); −0.008(1.5)
Example I-27: ¹H-NMR(400.0 MHz, CDCl3): δ =
8.107(2.3); 8.105(3.3); 8.103(4.2); 8.102(3.2); 8.099(2.0); 7.836(3.1); 7.832(2.9); 7.816(3.8); 7.812(3.6);
7.685(6.2); 7.679(6.1); 7.651(4.0); 7.650(3.8); 7.631(3.2); 7.630(3.1); 7.510(0.8); 7.494(0.8); 7.489(0.9);
7.485(0.9); 7.472(0.9); 7.468(0.9); 7.463(0.8); 7.446(0.8); 7.259(65.7); 7.176(13.5); 7.008(1.0); 6.992(1.1);
6.985(1.3); 6.969(1.3); 6.961(1.0); 6.944(1.0); 6.794(3.9); 6.792(4.0); 6.789(3.9); 6.786(3.7); 5.458(7.8);
5.298(1.1); 4.798(3.2); 1.540(16.0); 0.008(0.9); 0.000(28.3); −0.008(0.8)
Example I-28: ¹H-NMR(400.0 MHz, CDCl3): δ =
8.102(1.6); 8.100(2.4); 8.098(3.0); 8.097(2.4); 8.095(1.5); 7.813(2.3); 7.809(2.2); 7.793(2.7); 7.789(2.7);
7.676(4.8); 7.670(4.8); 7.634(3.0); 7.633(2.9); 7.613(2.4); 7.612(2.3); 7.264(0.5); 7.259(31.1); 7.246(0.7);
7.232(0.8); 7.228(0.8); 7.226(0.8); 7.225(0.8); 7.213(0.8); 7.210(0.9); 7.206(0.8); 7.192(0.8); 7.150(9.5);
6.947(1.1); 6.942(1.1); 6.925(1.0); 6.920(1.6); 6.916(1.1); 6.899(0.9); 6.894(0.9); 6.786(3.0); 6.784(3.1);
6.781(3.0); 6.778(3.0); 5.473(5.1); 5.471(5.2); 5.469(5.0); 5.297(0.7); 4.783(2.5); 4.018(8.7); 4.015(16.0);
4.012(8.6); 1.552(3.0); 0.000(13.6)
Example I-29: ¹H-NMR(400.0 MHz, CDCl3): δ =
8.397(4.9); 7.971(2.2); 7.950(8.2); 7.937(2.8); 7.933(5.0); 7.930(2.7); 7.912(1.5); 7.908(0.9); 7.518(2.4);
7.498(4.2); 7.494(6.8); 7.485(4.6); 7.480(9.7); 7.476(4.9); 7.468(1.7); 7.463(4.3); 7.414(5.8); 7.402(3.9);
7.259(419.7); 7.250(0.9); 7.102(0.7); 7.095(5.5); 7.090(1.7); 7.078(1.7); 7.073(10.0); 7.068(1.8); 7.057(1.6);
7.051(4.8); 6.995(2.2); 5.412(16.0); 4.876(4.1); 1.532(135.8); 1.264(0.8); 0.882(1.6); 0.865(0.6); 0.146(0.6);
0.008(5.1); 0.000(171.1); −0.008(4.6)
Example I-30: ¹H-NMR(400.0 MHz, CDCl3): δ =
8.102(3.9); 8.101(4.9); 8.099(3.8); 7.818(3.6); 7.814(3.4); 7.798(4.4); 7.794(4.3); 7.680(7.9); 7.675(7.8);
7.640(5.0); 7.639(4.8); 7.620(3.9); 7.466(0.7); 7.461(5.2); 7.460(4.4); 7.455(1.9); 7.444(2.5); 7.439(8.3);
7.433(1.2); 7.381(1.7); 7.375(10.8); 7.370(2.8); 7.359(2.3); 7.354(6.0); 7.348(0.6); 7.258(59.4); 7.153(13.9);
6.790(4.9); 6.787(5.0); 6.784(4.9); 6.782(4.6); 5.423(16.0); 5.296(11.6); 4.779(3.9); 1.544(8.7); 0.008(1.0);
0.000(28.0); −0.008(0.7)
Example I-31: ¹H-NMR(400.0 MHz, CDCl3): δ =
8.179(4.6); 8.176(4.5); 7.866(2.8); 7.861(2.7); 7.844(3.1); 7.840(3.0); 7.654(5.6); 7.648(5.6); 7.552(4.0);
7.530(3.4); 7.518(2.0); 7.458(5.4); 7.442(2.3); 7.437(8.2); 7.379(1.6); 7.373(9.9); 7.368(2.7); 7.357(2.3);
7.352(5.8); 7.292(0.5); 7.259(339.7); 7.251(1.2); 7.247(0.5); 7.209(0.7); 7.144(14.7); 7.037(0.6); 6.995(1.9);
6.820(4.1); 6.818(4.5); 6.815(4.5); 6.812(4.3); 5.422(16.0); 4.765(4.0); 1.532(55.4); 1.370(0.8); 1.333(1.7);
1.284(2.5); 1.256(1.3); 0.008(4.3); 0.000(132.2); −0.008(3.8)
Example I-32: ¹H-NMR(400.0 MHz, CDCl3): δ =
8.487(6.9); 8.485(7.4); 8.483(7.0); 8.481(3.7); 7.887(2.9); 7.884(2.7); 7.866(9.1); 7.862(9.2); 7.848(9.4);
7.847(9.4); 7.827(2.9); 7.826(2.7); 7.517(0.5); 7.503(13.0); 7.489(15.1); 7.346(7.2); 7.344(7.1); 7.332(6.1);
7.330(6.0); 7.258(84.1); 7.254(0.6); 7.208(0.6); 7.159(21.9); 6.755(0.6); 6.746(1.1); 6.738(5.3); 6.733(0.8);
6.720(6.6); 6.717(6.6); 6.704(0.8); 6.698(5.1); 6.691(1.0); 5.498(16.0); 5.296(10.0); 4.774(6.2); 1.555(10.4);
0.008(1.0); 0.000(37.4); −0.008(1.0)
Example I-33: ¹H-NMR(400.0 MHz, CDCl3): δ =
7.942(3.2); 7.925(3.3); 7.921(3.7); 7.905(3.5); 7.715(7.7); 7.710(7.8); 7.528(3.9); 7.518(5.4); 7.444(6.2);
7.423(5.8); 7.365(1.9); 7.357(2.2); 7.334(4.8); 7.325(7.4); 7.322(7.2); 7.320(4.8); 7.314(3.0); 7.310(4.8);
7.292(1.0); 7.290(1.8); 7.282(1.3); 7.259(985.9); 7.250(2.4); 7.237(0.8); 7.224(1.5); 7.209(0.8); 6.995(5.4);
6.837(4.0); 6.832(4.0); 6.829(4.3); 6.824(3.9); 5.428(16.0); 4.871(1.9); 1.534(20.7); 0.331(0.8); 0.157(1.2);
0.146(1.0); 0.008(11.2); 0.000(368.4); −0.008(9.8); −0.035(0.8); −0.150(1.0)
Example I-34: ¹H-NMR(400.0 MHz, CDCl3): δ =
8.173(2.7); 8.169(4.8); 8.166(2.8); 7.899(1.7); 7.895(3.2); 7.891(1.7); 7.878(1.9); 7.873(3.5); 7.869(1.8);
7.664(7.1); 7.658(7.0); 7.586(3.3); 7.584(4.8); 7.582(3.5); 7.564(3.0); 7.562(4.2); 7.560(3.0); 7.492(3.9);
7.486(1.6); 7.478(4.1); 7.477(3.3); 7.475(2.3); 7.473(2.2); 7.470(4.5); 7.462(1.7); 7.456(4.3); 7.449(0.5);
7.258(60.4); 7.098(0.6); 7.091(5.3); 7.086(1.6); 7.074(1.8); 7.069(9.8); 7.064(1.8); 7.053(1.7); 7.047(4.7);
6.833(5.5); 6.831(5.6); 6.828(5.6); 6.825(5.4); 5.404(16.0); 5.295(4.7); 4.864(4.0); 1.547(10.3); 0.008(0.7);
0.000(24.2); −0.008(0.7)
Example I-35: ¹H-NMR(400.0 MHz, CDCl3): δ =
7.694(0.6); 7.689(0.6); 7.258(12.0); 7.177(1.5); 7.156(1.5); 6.475(2.1); 6.470(2.2); 6.462(1.6); 6.456(0.7);
6.442(1.2); 6.436(0.9); 5.432(1.4); 4.620(1.8); 4.605(1.8); 3.850(3.4); 3.844(14.9); 3.813(3.7); 3.807(16.0);
3.780(0.7); 3.663(0.6); 2.118(0.6); 1.559(0.7); 0.000(5.1)
Example I-36: ¹H-NMR(400.0 MHz, CDCl3): δ =
8.174(5.7); 8.170(5.7); 7.862(3.6); 7.858(3.4); 7.841(4.1); 7.836(4.0); 7.666(2.9); 7.652(9.4); 7.646(16.0);
7.631(10.2); 7.610(2.4); 7.552(3.6); 7.551(5.0); 7.549(3.4); 7.531(3.2); 7.529(4.3); 7.258(42.9); 7.142(17.1);
6.813(5.5); 6.810(5.6); 6.807(5.5); 6.805(5.1); 5.509(15.1); 5.295(2.7); 4.791(5.3); 2.043(0.9); 1.558(4.7);
1.257(0.8); 0.008(0.6); 0.000(19.4); −0.008(0.6)
Example I-37: ¹H-NMR(400.0 MHz, CDCl3): δ =
8.199(8.3); 8.195(8.2); 7.886(5.4); 7.881(5.2); 7.864(6.1); 7.859(5.8); 7.836(3.8); 7.816(4.4); 7.752(0.6);
7.712(4.0); 7.692(4.6); 7.660(0.7); 7.651(11.2); 7.646(11.2); 7.605(2.1); 7.586(4.2); 7.567(2.4); 7.553(5.1);
7.551(7.3); 7.550(5.1); 7.532(4.4); 7.530(6.4); 7.528(4.5); 7.517(0.6); 7.460(2.5); 7.441(4.0); 7.421(1.6);
7.258(91.3); 7.208(0.8); 7.149(28.2); 6.817(8.3); 6.814(8.5); 6.811(8.3); 6.809(8.0); 5.672(16.0); 5.296(4.4);
4.786(7.4); 1.920(0.5); 1.902(0.5); 1.554(3.6); 1.282(0.5); 1.256(1.3); 0.008(1.2); 0.000(38.2); −0.008(1.1)
Example I-38: ¹H-NMR(400.0 MHz, CDCl3): δ =
8.198(4.5); 8.194(4.6); 7.874(2.7); 7.869(2.6); 7.852(3.1); 7.848(3.0); 7.648(5.7); 7.642(5.7); 7.549(7.5);
7.528(3.4); 7.382(0.8); 7.378(0.9); 7.369(2.0); 7.367(1.9); 7.359(1.9); 7.355(2.0); 7.349(0.6); 7.345(0.7);
7.341(0.6); 7.334(0.6); 7.330(0.7); 7.324(7.4); 7.322(6.7); 7.320(4.7); 7.314(2.7); 7.310(4.4); 7.309(4.3);
7.258(49.6); 7.143(13.3); 6.819(4.0); 6.817(4.3); 6.814(4.3); 6.811(4.1); 5.432(16.0); 5.296(1.4); 4.779(4.1);
1.547(2.6); 1.371(0.5); 1.333(0.5); 1.285(0.7); 1.282(0.8); 1.256(0.6); 0.008(0.6); 0.000(19.1); −0.008(0.6)
Example I-39: ¹H-NMR(400.0 MHz, CDCl3): δ =
9.045(0.6); 9.039(16.0); 8.625(5.5); 8.621(5.5); 8.179(4.5); 8.178(4.6); 8.157(6.0); 8.156(6.0); 8.023(5.0);
8.018(4.9); 8.001(3.8); 7.997(3.8); 7.588(1.2); 7.572(1.3); 7.568(2.5); 7.551(2.4); 7.546(1.5); 7.530(1.2);
7.518(1.0); 7.259(167.7); 7.199(16.9); 6.995(1.0); 6.938(1.0); 6.935(1.0); 6.931(1.1); 6.929(1.2); 6.915(1.6);
6.908(2.1); 6.896(1.0); 6.892(2.2); 6.887(1.9); 6.870(2.0); 6.868(2.0); 6.861(1.6); 6.845(1.7); 6.839(1.4);
5.489(11.4); 5.297(1.5); 4.833(4.1); 4.027(0.7); 1.557(8.4); 1.333(2.2); 1.283(3.0); 1.256(2.6); 0.880(0.5);
0.008(2.0); 0.000(62.9); −0.008(1.8)
Example I-40: ¹H-NMR(400.0 MHz, CDCl3): δ =
8.414(4.5); 8.411(3.8); 7.937(1.1); 7.916(7.0); 7.910(5.5); 7.906(5.0); 7.889(0.8); 7.885(0.8); 7.552(3.7);
7.550(3.6); 7.548(3.0); 7.476(4.7); 7.462(6.9); 7.403(5.2); 7.389(3.8); 7.383(1.0); 7.379(0.8); 7.374(2.0);
7.372(1.9); 7.364(2.0); 7.360(1.9); 7.354(0.6); 7.350(0.6); 7.344(0.6); 7.336(0.6); 7.332(0.6); 7.326(7.6);
7.324(6.5); 7.322(4.6); 7.317(3.1); 7.313(4.5); 7.312(4.8); 7.257(59.6); 7.191(12.6); 5.439(16.0); 5.295(3.3);
4.794(4.0); 1.546(4.9); 0.008(0.7); 0.000(22.5); −0.008(0.6)
Example I-41: ¹H-NMR(400.0 MHz, CDCl3): δ =
9.039(15.2); 8.644(5.2); 8.640(5.2); 8.183(4.4); 8.182(4.3); 8.162(5.8); 8.161(5.7); 8.034(4.8); 8.029(4.7);
8.012(3.6); 8.008(3.6); 7.518(0.6); 7.389(1.2); 7.374(1.3); 7.368(3.1); 7.354(2.4); 7.348(2.2); 7.334(1.8);
7.275(5.0); 7.268(2.9); 7.259(109.3); 7.252(2.3); 7.245(1.9); 7.210(16.1); 7.068(0.9); 7.065(1.0); 7.062(0.9);
7.059(0.9); 7.045(1.7); 7.040(1.6); 7.023(0.8); 7.019(0.8); 7.017(0.7); 6.995(0.6); 5.462(16.0); 5.297(2.4);
4.846(4.3); 4.026(0.5); 3.155(0.7); 1.559(4.0); 1.371(0.6); 1.334(1.7); 1.286(1.1); 1.282(2.5); 1.256(2.2);
0.008(1.2); 0.000(39.4); −0.008(1.2)
Example I-42: ¹H-NMR(400.0 MHz, CDCl3): δ =
8.494(6.8); 7.979(2.4); 7.975(4.3); 7.971(2.4); 7.958(4.1); 7.954(7.3); 7.950(4.2); 7.901(8.7); 7.881(4.9);
7.583(1.7); 7.567(1.9); 7.562(3.6); 7.547(15.7); 7.541(2.5); 7.534(15.4); 7.525(1.9); 7.517(2.9); 7.377(7.4);
7.375(7.5); 7.364(6.3); 7.362(6.4); 7.259(500.2); 7.251(1.6); 7.208(0.6); 6.994(2.7); 6.935(1.3); 6.932(1.4);
6.929(1.8); 6.926(1.6); 6.912(2.2); 6.905(2.9); 6.888(3.9); 6.884(2.1); 6.863(3.0); 6.857(2.3); 6.841(2.5);
6.835(2.0); 5.470(16.0); 4.881(5.9); 1.529(134.3); 1.306(0.8); 1.265(3.3); 0.899(1.8); 0.882(6.6); 0.864(2.4);
0.146(0.6); 0.008(6.1); 0.000(203.7); −0.008(5.4); −0.150(0.7)
Example I-43: ¹H-NMR(400.0 MHz, CDCl3): δ =
8.109(1.3); 8.107(1.9); 8.106(2.6); 8.104(1.9); 8.102(1.3); 7.822(2.0); 7.818(2.0); 7.801(2.5); 7.798(2.4);
7.681(4.0); 7.676(4.0); 7.642(2.4); 7.641(2.4); 7.622(2.0); 7.620(1.9); 7.601(0.5); 7.585(0.5); 7.580(1.0);
7.564(1.0); 7.559(0.6); 7.543(0.5); 7.518(0.5); 7.259(91.0); 7.209(0.6); 7.161(9.0); 6.919(0.6); 6.910(0.8);
6.892(0.5); 6.888(0.9); 6.881(0.6); 6.865(0.9); 6.862(0.8); 6.858(0.6); 6.856(0.7); 6.840(0.7); 6.834(0.6);
6.791(2.6); 6.789(2.6); 6.786(2.5); 6.783(2.4); 5.483(4.8); 5.298(3.2); 4.773(1.8); 1.536(16.0); 0.008(1.1);
0.000(38.0); −0.008(1.1)
Example I-44: ¹H-NMR(400.0 MHz, CDCl3): δ =
8.540(1.3); 8.538(2.3); 8.536(2.4); 8.534(2.3); 8.532(1.2); 7.932(1.4); 7.928(1.3); 7.911(2.8); 7.907(2.8);
7.874(2.6); 7.872(2.6); 7.853(1.3); 7.667(0.9); 7.661(0.8); 7.660(0.9); 7.652(0.6); 7.650(0.6); 7.649(0.8);
7.643(1.0); 7.518(0.6); 7.514(4.0); 7.501(4.7); 7.431(1.1); 7.426(0.7); 7.423(0.6); 7.415(1.2); 7.413(1.1);
7.408(1.5); 7.361(2.2); 7.359(2.2); 7.347(1.8); 7.345(1.8); 7.329(0.5); 7.315(1.8); 7.310(1.5); 7.308(1.7);
7.300(4.4); 7.292(1.5); 7.289(1.3); 7.284(1.2); 7.270(0.6); 7.269(0.5); 7.268(0.5); 7.267(0.6); 7.266(0.9);
7.265(1.0); 7.265(1.1); 7.264(1.3); 7.263(1.6); 7.259(88.5); 7.256(1.4); 7.255(0.8); 7.254(0.6); 7.209(0.7);
7.205(8.2); 6.995(0.5); 5.589(7.4); 5.588(7.4); 4.791(1.8); 2.044(0.6); 1.538(16.0); 1.326(0.5); 1.258(0.6);
0.882(0.8); 0.008(1.1); 0.000(39.3); −0.008(1.0)
Example I-45: ¹H-NMR(400.0 MHz, CDCl3): δ =
8.494(3.5); 8.492(3.9); 7.978(1.5); 7.974(2.5); 7.969(1.4); 7.957(2.6); 7.952(4.4); 7.948(2.5); 7.903(5.3);
7.902(5.3); 7.882(3.0); 7.880(2.8); 7.547(7.5); 7.534(8.6); 7.457(0.6); 7.451(4.9); 7.450(4.1); 7.446(1.8);
7.435(2.3); 7.429(8.2); 7.424(1.2); 7.380(2.0); 7.377(5.6); 7.375(13.9); 7.369(3.0); 7.364(4.0); 7.362(4.0);
7.358(2.3); 7.353(5.9); 7.347(0.6); 7.257(61.5); 5.412(16.0); 5.295(3.4); 4.884(3.6); 1.536(16.5); 0.008(0.8);
0.000(26.6); −0.008(0.7)
Example I-46: ¹H-NMR(400.0 MHz, CDCl3): δ =
8.495(7.0); 8.493(7.7); 8.491(7.4); 7.899(2.4); 7.896(2.2); 7.878(8.9); 7.875(9.1); 7.864(10.3); 7.863(10.3);
7.843(2.6); 7.515(13.5); 7.501(16.0); 7.357(7.8); 7.355(8.2); 7.347(1.5); 7.343(6.8); 7.342(7.0); 7.333(1.9);
7.332(1.8); 7.325(1.3); 7.321(1.2); 7.315(1.8); 7.313(1.9); 7.307(1.2); 7.299(1.1); 7.293(1.2); 7.267(0.7);
7.258(96.6); 7.254(0.6); 7.209(0.7); 7.188(20.4); 7.038(1.4); 7.032(1.4); 7.020(1.4); 7.015(3.0); 7.009(1.8);
6.997(1.8); 6.994(1.4); 6.992(2.8); 6.986(1.4); 6.974(1.2); 6.969(1.2); 5.495(14.1); 5.493(15.4); 5.492(13.9);
4.802(6.4); 1.555(14.8); 0.008(1.2); 0.000(43.6); −0.008(1.1)
Example I-47: ¹H-NMR(400.0 MHz, CDCl3): δ =
8.176(4.3); 7.902(2.5); 7.880(2.6); 7.669(7.4); 7.664(7.3); 7.590(4.1); 7.569(3.6); 7.518(3.4); 7.445(5.5);
7.429(2.7); 7.423(9.7); 7.375(2.0); 7.370(13.4); 7.364(3.4); 7.353(2.9); 7.348(7.2); 7.292(0.7); 7.271(2.1);
7.268(2.7); 7.266(4.2); 7.265(4.6); 7.259(626.6); 7.251(2.5); 7.251(2.2); 7.250(1.8); 7.247(1.2); 7.244(0.8);
7.228(0.7); 6.995(3.4); 6.841(5.1); 6.838(5.2); 6.835(5.1); 6.833(5.0); 5.405(16.0); 4.870(4.0); 1.530(136.4);
0.146(0.7); 0.008(7.4); 0.000(239.8); −0.008(6.4); −0.150(0.7)
Example I-48: ¹H-NMR(400.0 MHz, CDCl3): δ =
8.178(4.0); 8.175(7.1); 8.171(4.1); 7.904(2.4); 7.899(4.4); 7.895(2.5); 7.882(2.8); 7.878(5.1); 7.874(2.7);
7.666(10.3); 7.660(10.4); 7.588(4.9); 7.586(7.0); 7.584(5.0); 7.572(1.8); 7.566(4.4); 7.564(6.1); 7.562(4.4);
7.556(1.8); 7.551(3.5); 7.535(3.5); 7.530(2.1); 7.518(2.1); 7.514(2.0); 7.259(349.6); 6.995(1.9); 6.926(1.4);
6.923(1.4); 6.920(1.7); 6.917(1.8); 6.903(2.3); 6.896(3.2); 6.883(3.4); 6.877(2.9); 6.861(3.1); 6.858(3.1);
6.852(2.4); 6.836(10.5); 6.834(8.8); 6.831(9.4); 6.828(8.8); 5.461(16.0); 4.867(5.6); 4.130(0.8); 4.113(0.8);
2.043(3.6); 1.598(2.2); 1.276(1.1); 1.258(2.3); 1.240(1.1); 0.008(3.9); 0.000(134.4); −0.008(4.0)
Example I-49: ¹H-NMR(400.0 MHz, CDCl3): δ =
7.897(2.7); 7.881(2.8); 7.876(3.1); 7.860(2.9); 7.710(7.0); 7.705(7.1); 7.518(0.5); 7.446(5.0); 7.441(2.0);
7.428(8.2); 7.425(9.0); 7.419(1.4); 7.408(5.6); 7.373(1.6); 7.367(10.4); 7.362(2.8); 7.350(2.1); 7.346(5.9);
7.340(0.7); 7.300(6.3); 7.296(6.5); 7.259(90.1); 6.995(0.5); 6.832(3.5); 6.826(3.8); 6.824(3.9); 6.818(3.4);
5.413(16.0); 5.297(1.2); 4.830(3.2); 1.561(0.9); 1.476(0.6); 1.432(2.8); 1.428(1.0); 1.255(0.6); 0.070(2.0);
0.008(1.0); 0.000(34.0); −0.008(1.1)
Example I-50: ¹H-NMR(400.0 MHz, CDCl3): δ =
8.521(2.5); 8.519(4.4); 8.517(4.8); 8.515(4.5); 8.513(2.4); 7.916(2.4); 7.912(2.3); 7.895(5.5); 7.891(5.5);
7.865(5.6); 7.864(5.6); 7.844(2.4); 7.843(2.2); 7.554(1.6); 7.552(2.9); 7.550(3.6); 7.548(3.6); 7.546(2.9);
7.544(1.6); 7.510(7.9); 7.496(9.4); 7.390(0.9); 7.386(0.9); 7.379(2.3); 7.376(1.8); 7.373(1.2); 7.371(0.8);
7.368(2.0); 7.366(1.4); 7.364(2.0); 7.357(0.8); 7.354(4.8); 7.352(4.7); 7.349(0.7); 7.346(0.9); 7.340(3.9);
7.338(4.2); 7.334(0.6); 7.329(6.4); 7.327(8.0); 7.324(4.0); 7.318(3.3); 7.316(4.2); 7.314(6.2); 7.262(0.6);
7.262(0.7); 7.261(0.9); 7.260(1.2); 7.257(48.2); 7.253(0.5); 7.187(15.1); 5.440(16.0); 5.295(2.2); 4.795(3.9);
1.546(1.7); 0.071(5.4); 0.008(0.7); 0.000(22.7); −0.008(0.6)
Example I-51: ¹H-NMR(400.0 MHz, CDCl3): δ =
8.137(2.1); 7.906(1.0); 7.903(1.7); 7.899(1.0); 7.886(1.2); 7.882(2.2); 7.878(1.2); 7.714(4.1); 7.708(4.4);
7.698(0.9); 7.690(3.0); 7.670(2.3); 7.588(2.1); 7.568(2.5); 7.560(0.6); 7.518(1.8); 7.453(7.1); 7.442(3.3);
7.437(4.2); 7.433(9.3); 7.405(0.7); 7.385(10.6); 7.380(11.7); 7.352(0.6); 7.310(0.7); 7.304(1.9); 7.298(2.0);
7.287(7.9); 7.282(8.3); 7.277(3.0); 7.273(1.8); 7.272(2.0); 7.266(9.0); 7.266(7.6); 7.265(6.7); 7.259(319.4);
7.253(6.3); 7.252(5.7); 7.252(4.9); 7.251(4.3); 7.250(4.2; 7.249(3.9) 7.248(3.6); 7.248(3.5); 7.247(3.4);
7.246(3.3); 7.245(3.2); 7.244(3.0); 7.242(2.8); 7.241(2.8); 7.231(7.8); 7.209(1.5); 6.995(1.8); 6.974(0.9);
6.970(1.3); 6.825(0.5); 6.818(2.9); 6.815(2.8); 6.812(3.2); 6.810(3.0); 5.510(8.5); 5.136(0.6); 4.896(2.2);
4.766(15.6); 4.750(16.0); 4.723(0.8); 2.043(0.5); 1.888(6.0); 1.873(12.3); 1.857(6.1); 1.533(79.6); 1.505(2.3);
1.265(1.3); 0.899(0.7); 0.882(2.3); 0.864(0.9); 0.008(4.2); 0.000(140.3); −0.008(5.0); −0.028(3.2);
−0.050(0.7)
Example I-52: ¹H-NMR(400.0 MHz, CDCl3): δ =
8.172(3.8); 8.169(6.6); 8.165(3.9); 7.897(2.2); 7.892(4.3); 7.888(2.4); 7.875(2.6); 7.871(4.7); 7.866(2.5);
7.674(0.8); 7.669(1.2); 7.666(1.2); 7.658(9.6); 7.652(9.6); 7.577(4.7); 7.575(6.3); 7.573(4.5); 7.555(4.0);
7.553(5.8); 7.551(4.0); 7.517(0.7); 7.458(0.5); 7.421(0.5); 7.258(105.9); 6.994(0.6); 6.852(0.6); 6.850(0.6);
6.847(0.6); 6.844(0.6); 6.829(7.2); 6.827(7.3); 6.823(7.4); 6.821(7.0); 6.750(0.6); 6.742(1.1); 6.734(5.5);
6.729(1.4); 6.726(1.3); 6.724(1.0); 6.716(7.1); 6.712(7.2); 6.700(1.0); 6.694(5.2); 6.686(1.4); 6.678(0.6);
6.416(0.8); 5.474(16.0); 5.437(0.9); 5.296(15.7); 5.045(0.9); 5.011(0.6); 4.853(5.4); 1.539(19.3); 0.008(1.4);
0.000(45.3); −0.008(1.2)
Example I-53: ¹H-NMR(400.0 MHz, CDCl3): δ =
8.488(4.8); 7.973(1.7); 7.969(2.9); 7.965(1.6); 7.952(3.0); 7.948(5.1); 7.944(2.9); 7.898(6.2); 7.877(3.5);
7.544(8.9); 7.530(10.3); 7.500(4.0); 7.494(1.6); 7.486(4.2); 7.478(4.4); 7.470(1.7); 7.464(4.3); 7.374(5.2);
7.372(5.1); 7.360(4.5); 7.358(4.4); 7.257(56.1); 7.105(0.6); 7.098(5.4); 7.092(1.6); 7.081(1.8); 7.076(10.0);
7.070(1.8); 7.059(1.6); 7.054(4.8); 5.413(16.0); 5.294(5.2); 4.877(4.2); 1.541(3.3); 0.008(0.7); 0.000(24.3);
−0.008(0.7)
Example I-54: ¹H-NMR(400.0 MHz, CDCl3): δ =
8.092(2.0); 8.090(2.9); 8.089(3.7); 8.087(2.8); 8.085(1.9); 7.813(2.8); 7.810(2.6); 7.793(3.5); 7.789(3.4);
7.681(5.5); 7.676(5.5); 7.641(3.6); 7.640(3.5); 7.620(2.9); 7.259(72.1); 7.164(12.2); 6.790(3.5); 6.787(3.5);
6.784(3.4); 6.782(3.3); 5.537(4.1); 5.533(7.1); 5.530(3.9); 4.787(3.0); 1.537(16.0); 0.008(1.0); 0.000(31.9);
−0.008(0.9)
Example I-55: ¹H-NMR(400.0 MHz, CDCl3): δ =
8.484(5.8); 8.483(5.2); 7.970(2.2); 7.965(3.6); 7.961(2.1); 7.949(3.6); 7.944(6.3); 7.940(3.6); 7.890(7.3);
7.889(7.4); 7.869(4.3); 7.868(4.2); 7.539(11.4); 7.525(13.2); 7.517(0.6); 7.368(6.4); 7.366(6.4); 7.354(5.5);
7.352(5.5); 7.258(109.2); 6.994(0.6); 6.747(1.0); 6.740(4.9); 6.734(0.8); 6.721(6.2); 6.718(6.2); 6.705(0.8);
6.700(4.8); 6.692(1.0); 5.482(14.4); 5.296(16.0); 4.867(5.1); 1.537(32.2); 0.008(1.3); 0.000(43.5); −0.008(1.2)
Example I-56: ¹H-NMR(400.0 MHz, CDCl3): δ =
8.400(6.7); 7.971(3.0); 7.949(11.7); 7.948(6.9); 7.939(4.1); 7.935(7.2); 7.931(3.7); 7.914(2.0); 7.578(1.7);
7.561(1.8); 7.557(3.5); 7.541(3.4); 7.535(1.9); 7.518(4.1); 7.493(8.0); 7.479(11.3); 7.414(8.1); 7.400(5.8);
7.382(0.6); 7.342(0.5); 7.259(581.0); 7.228(0.6); 7.210(0.6); 6.995(3.3); 6.925(1.4); 6.922(1.7); 6.919(1.6);
6.905(2.2); 6.899(3.0); 6.885(3.6); 6.878(3.0); 6.860(3.0); 6.854(2.2); 6.838(2.6); 6.832(2.1); 5.468(16.0);
4.878(5.8); 1.532(172.4); 0.146(0.6); 0.008(7.2); 0.000(237.4); −0.008(6.6); −0.150(0.7)
Example I-57: ¹H-NMR(400.0 MHz, CDCl3): δ =
8.147(2.3); 8.143(2.3); 7.837(1.2); 7.832(1.2); 7.815(1.4); 7.811(1.4); 7.643(2.6); 7.637(2.6); 7.525(1.9);
7.504(1.6); 7.355(2.4); 7.334(3.0); 7.259(31.3); 7.176(2.9); 7.155(2.3); 7.117(5.5); 6.799(2.0); 6.793(2.0);
5.627(9.2); 5.297(4.9); 4.748(2.5); 3.995(16.0); 1.546(4.3); 0.000(9.9)
Example I-58: ¹H-NMR(400.0 MHz, CDCl3): δ =
8.027(5.0); 8.011(5.0); 7.660(3.7); 7.635(3.6); 7.505(0.5); 7.437(6.4); 7.423(11.4); 7.421(5.1); 7.417(2.1);
7.406(2.5); 7.401(9.2); 7.395(1.5); 7.360(2.3); 7.357(5.7); 7.355(13.9); 7.349(3.4); 7.344(3.6); 7.342(3.6);
7.338(2.4); 7.333(6.0); 7.327(0.7); 7.266(0.7); 7.265(0.7); 7.258(73.8); 5.393(16.0); 5.296(4.3); 4.911(3.8);
1.539(22.9); 1.257(0.6); 0.008(0.9); 0.000(30.6); −0.008(0.8)
Example I-59: ¹H-NMR(400.0 MHz, CDCl3): δ =
8.397(6.1); 8.395(6.4); 7.961(2.5); 7.940(11.1); 7.938(6.6); 7.931(4.2); 7.928(7.0); 7.924(3.7); 7.910(0.9);
7.906(1.6); 7.903(0.9); 7.517(1.4); 7.485(7.4); 7.471(10.7); 7.410(7.5); 7.408(7.9); 7.396(5.3); 7.395(5.4);
7.258(243.0); 6.994(1.3); 6.753(0.6); 6.745(1.1); 6.737(5.4); 6.718(6.8); 6.715(6.9); 6.702(0.9); 6.697(5.3);
6.689(1.1); 6.681(0.5); 5.480(16.0); 4.865(5.6); 1.534(96.9); 0.008(3.0); 0.000(97.0); −0.008(2.6)
Example I-60: ¹H-NMR(400.0 MHz, CDCl3): δ =
9.044(11.9); 8.659(3.7); 8.657(4.1); 8.654(4.0); 8.653(3.9); 8.189(3.4); 8.188(3.5); 8.168(4.5); 8.166(4.6);
8.040(3.9); 8.035(3.9); 8.018(3.0); 8.014(3.0); 7.560(1.3); 7.556(3.0); 7.554(2.8); 7.552(2.5); 7.518(1.1);
7.388(0.7); 7.384(0.7); 7.379(0.5); 7.377(1.0); 7.375(1.6); 7.372(1.6); 7.365(1.8); 7.361(1.8); 7.354(0.7);
7.353(0.9); 7.351(0.7); 7.346(0.6); 7.341(0.6); 7.336(6.9); 7.334(5.9); 7.332(4.1); 7.326(2.3); 7.323(3.7);
7.321(3.4); 7.275(0.5); 7.274(0.5); 7.273(0.6); 7.272(0.6); 7.271(0.7); 7.271(0.8); 7.270(0.9); 7.269(1.0);
7.268(1.2); 7.267(1.3); 7.267(1.5); 7.266(1.8); 7.265(2.1); 7.264(2.7); 7.263(3.6); 7.259(199.4); 7.255(3.2);
7.254(2.1); 7.254(1.4); 7.253(1.0); 7.252(0.8); 7.251(0.6); 7.250(0.6); 7.234(0.6); 7.230(10.8); 7.226(0.6);
6.995(1.1); 5.447(13.6); 4.888(2.0); 4.148(1.1); 4.130(3.4); 4.113(3.4); 4.095(1.1); 2.050(0.6); 2.043(16.0);
1.598(0.6); 1.283(0.7); 1.276(5.2); 1.258(10.5); 1.241(4.9); 0.008(2.2); 0.006(0.6); 0.000(81.2); −0.005(1.0);
−0.006(0.8); −0.007(0.7); −0.008(2.3)
Example I-61: ¹H-NMR(400.0 MHz, CDCl3): δ =
8.512(1.5); 8.510(2.7); 8.508(2.9); 8.506(2.7); 8.504(1.4); 7.920(1.1); 7.916(1.0); 7.899(3.6); 7.895(3.7);
7.881(3.5); 7.880(3.5); 7.860(1.0); 7.859(1.0); 7.525(4.8); 7.518(0.6); 7.511(5.6); 7.366(2.7); 7.364(2.6);
7.353(2.3); 7.351(2.2); 7.324(0.5); 7.320(0.6); 7.305(0.5); 7.299(0.6); 7.259(83.1); 7.255(0.7); 7.220(9.6);
7.209(0.5); 5.488(4.3); 5.487(5.2); 5.485(4.3); 4.823(2.1); 1.543(16.0); 0.008(1.1); 0.004(0.5); 0.002(1.4);
0.000(34.9); −0.008(1.0)
Example I-62: ¹H-NMR(400.0 MHz, CDCl3): δ =
8.226(5.5); 8.222(5.7); 8.196(3.2); 8.193(3.4); 8.175(3.4); 8.172(3.5); 7.961(2.7); 7.942(3.2); 7.916(3.2);
7.911(3.2); 7.894(3.6); 7.889(3.6); 7.711(1.8); 7.692(3.4); 7.673(2.4); 7.665(6.8); 7.660(6.5); 7.574(5.0);
7.552(4.0); 7.531(1.9); 7.518(3.5); 7.492(1.4); 7.259(521.2); 7.223(0.9); 7.191(15.3); 6.995(2.8); 6.836(5.1);
6.832(4.8); 6.830(4.8); 5.901(16.0); 5.523(1.0); 5.298(1.9); 4.806(5.2); 1.530(124.6); 1.332(0.8); 1.284(1.2);
1.256(1.0); 0.145(0.7); 0.008(6.5); 0.000(189.9); −0.008(5.8); −0.150(0.7)
Example I-63: ¹H-NMR(400.0 MHz, CDCl3): δ =
8.199(2.4); 8.195(4.1); 8.192(2.4); 7.926(1.5); 7.922(2.7); 7.918(1.5); 7.905(1.6); 7.900(3.0); 7.896(1.6);
7.671(6.1); 7.666(6.1); 7.597(3.0); 7.595(4.1); 7.593(3.1); 7.576(2.8); 7.573(5.6); 7.572(6.6); 7.551(4.5);
7.517(0.5); 7.440(5.3); 7.435(5.6); 7.290(3.7); 7.284(3.6); 7.269(3.6); 7.264(4.1); 7.258(97.8); 7.252(0.6);
6.994(0.5); 6.842(4.9); 6.840(5.0); 6.837(4.9); 6.834(4.8); 5.507(16.0); 4.884(3.6); 1.533(21.7); 0.008(1.3);
0.000(44.2); −0.008(1.1)
Example I-64: ¹H-NMR(400.0 MHz, CDCl3): δ =
9.071(14.0); 9.036(1.8); 8.585(4.5); 8.242(0.7); 8.237(0.7); 8.231(0.8); 8.222(4.3); 8.201(5.9); 8.200(5.8);
8.132(2.3); 8.127(4.4); 8.123(2.3); 8.110(1.5); 8.106(2.8); 8.101(1.4); 7.833(0.6); 7.828(0.6); 7.807(0.5);
7.518(0.9); 7.447(4.8); 7.442(1.8); 7.431(2.3); 7.426(8.6); 7.420(1.3); 7.382(1.7); 7.376(11.0); 7.371(3.0);
7.360(2.2); 7.355(5.8); 7.349(0.9); 7.345(1.7); 7.339(0.8); 7.329(1.4); 7.323(7.3); 7.319(1.9); 7.310(5.4);
7.304(1.0); 7.294(0.6); 7.288(1.5); 7.259(160.5); 6.995(0.9); 5.414(16.0); 4.925(3.7); 4.676(6.1); 3.016(1.3);
1.552(2.7); 1.370(0.8); 1.286(1.2); 1.255(0.8); 0.008(1.8); 0.000(59.7); −0.008(1.6)
Example I-65: ¹H-NMR(400.0 MHz, CDCl3): δ =
8.399(4.1); 7.974(2.2); 7.954(4.0); 7.953(7.3); 7.951(4.1); 7.940(2.4); 7.936(4.2); 7.932(2.3); 7.919(0.6);
7.914(1.2); 7.911(0.7); 7.517(1.5); 7.496(4.7); 7.482(6.7); 7.449(5.1); 7.444(1.9); 7.432(2.5); 7.427(8.7);
7.422(1.5); 7.418(5.0); 7.417(5.1); 7.405(3.5); 7.403(3.5); 7.377(1.7); 7.372(11.2); 7.366(3.0); 7.355(2.3);
7.350(6.1); 7.344(0.7); 7.259(274.2); 6.995(1.5); 5.411(16.0); 4.882(3.6); 2.043(1.4); 1.531(81.3); 1.276(0.5);
1.258(1.0); 0.008(3.4); 0.000(113.4); −0.008(3.0)
Example I-66: ¹H-NMR(400.0 MHz, CDCl3): δ =
7.797(8.8); 7.780(8.7); 7.653(11.8); 7.647(11.9); 7.543(1.7); 7.527(1.9); 7.522(3.5); 7.518(1.0); 7.506(3.5)
; 7.501(2.1); 7.485(1.9); 7.319(5.3); 7.318(5.4); 7.295(5.2); 7.294(5.3); 7.293(5.5); 7.267(0.9); 7.266(1.0);
7.259(110.0); 6.995(0.6); 6.910(1.4); 6.908(1.4); 6.904(1.8); 6.901(1.8); 6.889(2.1); 6.887(2.3); 6.881(3.3);
6.869(3.4); 6.863(2.6); 6.860(2.1); 6.848(3.0); 6.846(3.0); 6.842(2.0); 6.839(2.3); 6.823(2.7); 6.817(2.2);
6.799(8.1); 6.797(8.3); 6.794(8.3); 6.791(8.0); 5.447(16.0); 5.296(2.0); 4.896(5.9); 1.543(22.2); 1.257(0.6);
0.008(1.4); 0.000(44.2); −0.008(1.2)
Example I-67: ¹H-NMR(400.0 MHz, CDCl3): δ =
8.108(1.0); 8.106(1.4); 8.105(1.8); 8.103(1.3); 8.101(1.0); 7.838(1.5); 7.834(1.4); 7.817(1.8); 7.814(1.8);
7.690(2.8); 7.684(2.8); 7.658(1.7); 7.657(1.7); 7.638(1.4); 7.636(1.4); 7.259(76.5); 7.255(0.5); 7.192(6.8);
6.799(1.9); 6.797(1.9); 6.794(1.9); 6.791(1.8); 5.482(2.6); 5.480(3.2); 5.478(2.7); 4.810(1.3); 1.533(16.0);
0.008(1.1); 0.004(0.6); 0.002(1.5); 0.000(33.4); −0.003(1.4); −0.008(1.0)
Example I-68: ¹H-NMR(400.0 MHz, CDCl3): δ =
7.797(5.3); 7.779(5.3); 7.657(7.1); 7.651(7.2); 7.518(1.0); 7.427(0.7); 7.421(4.9); 7.420(4.0); 7.416(1.8);
7.405(2.4); 7.400(9.0); 7.394(1.5); 7.361(1.9); 7.355(11.6); 7.350(3.0); 7.339(2.2); 7.334(5.6); 7.324(3.3);
7.322(3.3); 7.299(3.2); 7.298(3.4); 7.259(179.7); 6.995(1.0); 6.804(4.9); 6.802(5.0); 6.798(5.0); 6.796(4.9);
5.390(16.0); 4.898(3.6); 1.535(43.9); 1.255(0.6); 0.008(2.1); 0.000(70.1); −0.008(2.0)
Example I-69: ¹H-NMR(400.0 MHz, CDCl3): δ =
9.040(10.6); 8.622(3.4); 8.621(3.6); 8.618(3.6); 8.616(3.4); 8.179(3.1); 8.178(3.1); 8.158(4.1); 8.156(4.1);
8.022(3.5); 8.017(3.4); 8.000(2.7); 7.996(2.7); 7.459(3.9); 7.458(3.2); 7.454(1.4); 7.443(1.8); 7.438(6.3);
7.432(1.0); 7.385(1.2); 7.379(8.0); 7.374(2.2); 7.362(1.9); 7.357(4.8); 7.352(0.8); 7.259(78.8); 7.254(0.5);
7.199(12.0); 5.431(11.8); 5.297(16.0); 4.839(2.8); 1.427(0.5); 1.422(0.8); 1.371(1.3); 1.333(8.5); 1.286(2.7);
1.282(12.0); 1.256(6.4); 1.232(0.8); 0.880(1.4); 0.862(0.6); 0.008(0.9); 0.000(32.7); −0.008(1.0)
Example I-70: ¹H-NMR(400.0 MHz, CDCl3): δ =
9.073(13.3); 8.606(4.7); 8.229(3.7); 8.228(3.6); 8.207(6.1); 8.206(6.1); 8.149(2.5); 8.145(4.6); 8.141(2.4);
8.128(1.5); 8.123(2.7); 8.119(1.4); 7.518(1.7); 7.387(1.2); 7.372(1.8); 7.367(3.1); 7.352(2.5); 7.346(2.0);
7.333(1.8); 7.292(0.7); 7.259(316.7); 7.247(3.8); 7.232(1.5); 7.227(1.6); 7.225(1.4); 7.066(1.0); 7.062(1.0);
7.045(1.8); 7.040(1.5); 7.024(0.9); 7.020(0.8); 6.995(1.7); 5.446(16.0); 4.934(4.0); 1.536(65.6); 1.333(1.0);
1.284(1.6); 1.256(1.8); 0.008(3.8); 0.000(123.8); −0.008(3.4)
Example I-71: ¹H-NMR(400.0 MHz, CDCl3): δ =
7.794(6.1); 7.777(6.1); 7.654(8.2); 7.648(8.3); 7.471(4.0); 7.466(1.6); 7.458(4.3); 7.455(2.2); 7.449(4.6);
7.442(1.8); 7.436(4.4); 7.429(0.5); 7.320(3.7); 7.319(3.8); 7.296(3.8); 7.294(3.8); 7.268(0.5); 7.266(0.8);
7.258(86.2); 7.086(0.7); 7.078(5.5); 7.073(1.7); 7.069(0.6); 7.062(1.8); 7.056(10.0); 7.051(1.8); 7.040(1.6);
7.035(4.8); 7.027(0.5); 6.799(5.7); 6.796(5.8); 6.793(5.8); 6.791(5.7); 5.391(16.0); 5.296(1.4); 4.894(4.1);
1.543(18.2); 1.256(0.6); 0.008(1.0); 0.000(34.6); −0.008(0.9)
Example I-72: ¹H-NMR(400.0 MHz, CDCl3): δ =
9.045(16.0); 8.635(5.2); 8.634(5.8); 8.631(5.6); 8.629(5.4); 8.190(4.8); 8.189(4.9); 8.169(6.3); 8.168(6.3);
8.037(5.4); 8.033(5.3); 8.016(4.2); 8.011(4.1); 7.676(2.8); 7.655(9.8); 7.638(8.6); 7.617(2.5); 7.518(1.2);
7.259(205.7); 7.250(0.5); 7.220(18.6); 6.995(1.2); 5.521(13.5); 4.850(4.5); 4.027(0.8); 3.227(1.0); 1.548(21.2);
1.333(3.3); 1.283(4.6); 1.257(3.6); 0.880(0.8); 0.008(2.4); 0.000(83.2); −0.008(2.4)
Example I-73: ¹H-NMR(400.0 MHz, CDCl3): δ =
8.417(5.1); 8.414(4.9); 7.952(1.7); 7.950(3.2); 7.948(1.7); 7.930(4.6); 7.929(8.3); 7.927(4.6); 7.906(5.0);
7.902(4.6); 7.885(1.8); 7.881(1.9); 7.518(1.0); 7.492(5.3); 7.478(7.5); 7.413(5.5); 7.411(5.6); 7.399(4.0);
7.398(3.9); 7.359(0.7); 7.354(0.7); 7.341(1.4); 7.335(1.3); 7.326(1.0); 7.319(0.9); 7.313(1.2); 7.309(0.8);
7.300(0.8); 7.294(0.8); 7.273(0.6); 7.272(0.6); 7.271(0.6); 7.270(0.7); 7.270(0.8); 7.269(0.9); 7.268(1.0);
7.267(1.2); 7.266(1.3); 7.266(1.5); 7.265(1.8); 7.264(2.3); 7.263(3.2); 7.259(172.2); 7.254(2.2); 7.253(1.6);
7.252(1.2); 7.252(1.0); 7.251(0.8); 7.250(0.6); 7.249(0.5); 7.222(21.1); 7.209(1.1); 6.995(1.0); 5.489(8.8);
5.487(10.8); 5.485(9.4); 5.298(0.6); 4.819(4.5); 1.537(16.0); 1.258(0.5); 0.008(2.0); 0.000(75.0); −0.008(2.2)
Example I-74: ¹H-NMR(400.0 MHz, CDCl3): δ =
8.394(2.2); 8.377(1.0); 8.373(1.0); 8.348(2.8); 8.345(2.6); 7.946(0.8); 7.925(3.2); 7.906(2.2); 7.904(3.7);
7.902(2.1); 7.886(0.9); 7.882(0.9); 7.864(0.6); 7.857(2.3); 7.854(2.2); 7.836(1.2); 7.832(1.2); 7.518(0.7);
7.480(3.9); 7.467(5.5); 7.405(1.4); 7.404(1.4); 7.398(3.0); 7.397(3.0); 7.392(1.0); 7.390(1.0); 7.385(2.1);
7.383(2.1); 7.259(119.0); 7.237(0.7); 7.223(0.8); 7.216(0.9); 7.209(0.8); 7.201(0.9); 7.197(0.8); 7.182(0.8);
7.123(3.0); 7.044(6.5); 6.995(0.9); 6.961(0.5); 6.953(0.9); 6.939(2.0); 6.934(1.6); 6.920(1.1); 6.916(2.2);
6.912(2.8); 6.908(1.2); 6.891(2.7); 6.886(2.1); 6.869(0.6); 6.864(0.7); 5.472(5.1); 5.429(0.6); 5.414(1.2);
5.398(0.6); 4.588(2.8); 4.573(2.6); 4.029(8.6); 4.027(12.4); 4.024(8.2); 4.012(8.6); 4.010(16.0); 4.007(8.5);
1.545(3.4); 1.255(1.0); 0.008(1.4); 0.000(51.0); −0.008(1.4)
Example I-75: ¹H-NMR(400.0 MHz, CDCl3): δ =
8.029(8.2); 8.013(8.2); 7.732(0.6); 7.730(0.6); 7.657(6.0); 7.632(6.0); 7.545(1.8); 7.529(1.9); 7.525(3.7);
7.517(1.0); 7.509(3.6); 7.504(2.9); 7.488(2.0); 7.435(10.5); 7.421(14.8); 7.356(7.6); 7.354(7.9); 7.342(5.5);
7.340(5.5); 7.269(0.7); 7.269(0.7); 7.268(0.7); 7.267(0.7); 7.265(1.3); 7.264(1.5); 7.258(132.2); 7.251(0.5);
6.994(0.7); 6.911(1.5); 6.908(1.5); 6.904(1.8); 6.902(1.9); 6.890(2.2); 6.888(2.3); 6.883(3.0); 6.881(3.5);
6.870(3.3); 6.863(2.5); 6.860(2.2); 6.848(3.0); 6.846(3.2); 6.842(2.1); 6.840(2.4); 6.824(2.5); 6.818(2.0);
5.451(16.0); 5.296(1.5); 4.907(6.3); 1.540(36.8); 1.258(0.7); 0.008(1.4); 0.000(53.5); −0.008(1.5)
Example I-76: ¹H-NMR(400.0 MHz, CDCl3): δ =
8.520(2.6); 8.518(3.5); 8.517(3.9); 8.001(1.6); 7.996(2.8); 7.992(1.6); 7.979(2.5); 7.975(4.5); 7.971(2.5);
7.912(5.2); 7.910(5.2); 7.890(3.2); 7.889(3.1); 7.588(3.9); 7.567(4.5); 7.553(7.9); 7.540(9.2); 7.517(0.7);
7.445(5.4); 7.440(5.8); 7.383(4.5); 7.381(4.5); 7.369(3.8); 7.368(3.8); 7.302(3.6); 7.297(3.4); 7.281(3.2);
7.276(3.1); 7.258(123.4); 6.994(0.7); 5.515(16.0); 4.900(3.8); 1.531(35.1); 0.008(1.7); 0.000(52.5); −0.008(1.4)
Example I-77: ¹H-NMR(400.0 MHz, CDCl3): δ =
8.027(5.4); 8.011(5.4); 7.658(4.0); 7.634(4.0); 7.517(0.7); 7.505(0.6); 7.503(0.6); 7.492(0.6); 7.481(0.5);
7.474(3.8); 7.469(1.7); 7.461(4.0); 7.458(2.4); 7.455(2.2); 7.452(4.5); 7.444(1.7); 7.439(4.6); 7.436(7.8);
7.422(9.7); 7.355(5.2); 7.353(5.4); 7.341(3.8); 7.340(3.8); 7.266(0.8); 7.258(87.9); 7.086(0.6); 7.079(5.3);
7.074(1.7); 7.062(1.7); 7.057(9.7); 7.052(1.8); 7.041(1.5); 7.035(4.6); 5.396(16.0); 5.296(2.8); 4.904(4.4);
1.538(25.2); 1.258(0.6); 0.008(1.2); 0.000(36.6); −0.008(1.0)
Example I-78: ¹H-NMR(400.0 MHz, CDCl3): δ =
9.051(15.7); 9.039(1.8); 8.648(4.8); 8.647(5.2); 8.644(5.1); 8.643(4.9); 8.586(0.6); 8.401(1.0); 8.194(4.9);
8.193(4.6); 8.173(6.5); 8.171(6.0); 8.045(5.0); 8.040(4.9); 8.023(4.0); 8.019(4.1); 8.003(0.6); 7.998(0.6);
7.590(4.1); 7.569(4.7); 7.520(3.1); 7.453(5.8); 7.448(6.2); 7.312(0.7); 7.307(3.9); 7.302(3.5); 7.286(3.7);
7.281(3.7); 7.270(3.4); 7.262(553.5); 7.255(2.3); 7.254(1.9); 7.253(1.4); 7.252(0.9); 7.252(0.9); 7.251(0.9);
7.250(0.8); 7.249(0.7); 7.248(0.6); 7.248(0.6); 7.247(0.6); 7.234(0.6); 7.225(17.8); 7.212(1.1); 7.136(1.9);
6.998(3.0); 5.537(16.0); 5.302(2.0); 4.849(4.2); 1.551(125.9); 1.333(2.6); 1.284(3.8); 1.255(2.0); 0.882(0.7);
0.146(0.6); 0.008(6.5); 0.000(207.3); −0.006(1.5); −0.007(1.3); −0.008(5.5); −0.150(0.6)
Example I-79: ¹H-NMR(400.0 MHz, CDCl3): δ =
7.798(8.3); 7.780(8.2); 7.648(11.1); 7.642(11.2); 7.518(0.7); 7.309(5.2); 7.308(5.1); 7.285(4.9); 7.283(5.2);
7.270(0.6); 7.268(0.7); 7.259(116.9); 6.995(0.6); 6.796(7.8); 6.794(8.0); 6.791(7.9); 6.788(7.7); 6.739(0.6);
6.731(1.1); 6.723(5.5); 6.717(1.0); 6.705(6.8); 6.702(6.9); 6.689(0.8); 6.683(5.2); 6.676(1.1); 5.462(16.0);
5.297(4.8); 4.882(5.6); 2.042(0.6); 1.541(36.3); 1.258(0.9); 0.008(1.4); 0.000(46.7); −0.008(1.3)
Example I-80: ¹H-NMR(400.0 MHz, CDCl3): δ =
8.045(4.9); 8.028(4.9); 7.669(3.8); 7.645(3.7); 7.535(4.2); 7.518(6.2); 7.514(5.2); 7.445(6.5); 7.431(10.1);
7.424(6.6); 7.378(1.0); 7.364(4.8); 7.352(3.6); 7.309(1.3); 7.274(5.9); 7.269(7.4); 7.259(1013.5); 7.249(4.5);
6.995(5.4); 5.499(16.0); 4.923(4.1); 1.530(299.2); 0.146(1.3); 0.008(12.5); 0.000(401.7); −0.008(10.7);
−0.150(1.2)
Example I-81: ¹H-NMR(400.0 MHz, CDCl3): δ =
7.811(5.5); 7.794(5.4); 7.661(7.4); 7.655(7.5); 7.529(4.0); 7.518(2.7); 7.509(4.7); 7.428(6.1); 7.423(6.5);
7.330(3.4); 7.307(3.4); 7.305(3.4); 7.274(4.9); 7.269(5.9); 7.259(480.7); 7.248(3.6); 6.995(2.5); 6.809(5.1);
6.807(5.2); 6.804(5.2); 6.801(5.2); 5.495(16.0); 4.911(3.8); 1.532(126.2); 1.258(0.6); 0.146(0.5); 0.008(5.8);
0.000(185.6); −0.008(4.8); −0.150(0.6)
Example I-82: ¹H-NMR(400.0 MHz, CDCl3): δ =
8.423(4.0); 8.421(4.2); 7.982(0.9); 7.961(7.2); 7.959(6.9); 7.955(5.6); 7.952(3.0); 7.934(0.7); 7.580(4.0);
7.559(4.6); 7.517(1.0); 7.500(5.2); 7.486(7.3); 7.443(5.9); 7.438(6.2); 7.420(5.6); 7.407(3.8); 7.293(3.7);
7.287(3.6); 7.272(3.7); 7.266(4.0); 7.258(185.5); 7.250(0.8); 7.249(0.6); 6.994(1.0); 5.513(16.0); 4.898(3.8);
1.532(59.6); 0.008(2.3); 0.000(74.0); −0.008(2.1)
Example I-83: ¹H-NMR(400.0 MHz, CDCl3): δ =
9.071(14.2); 8.584(4.8); 8.221(4.0); 8.220(4.0); 8.200(6.4); 8.198(6.3); 8.131(2.6); 8.126(4.8); 8.122(2.5);
8.109(1.7); 8.105(3.1); 8.100(1.6); 7.518(1.8); 7.498(3.6); 7.493(1.5); 7.485(3.8); 7.476(4.2); 7.468(1.6);
7.463(4.0); 7.259(318.6); 7.108(0.5); 7.100(4.9); 7.095(1.5); 7.084(1.7); 7.079(9.1); 7.074(1.6); 7.062(1.4);
7.057(4.3); 6.995(1.8); 5.417(16.0); 5.298(1.4); 4.917(4.2); 1.536(33.0); 1.370(0.8); 1.333(1.0); 1.284(1.7);
1.256(1.7); 0.008(3.8); 0.000(120.5); −0.008(3.3)

### B. Formulation Examples

1. Dusting Products

A dusting product is obtained by mixing 10 parts by weight of a compound of the general formula (I) and 90 parts by weight of talc as an inert substance and comminuting the mixture in a hammer mill.

2. Dispersible Powder

A readily water-dispersible wettable powder is obtained by mixing 25 parts by weight of a compound of the general formula (I), 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill.

3. Dispersion Concentrate

A readily water-dispersible dispersion concentrate is obtained by mixing 20 parts by weight of a compound of the general formula (I), 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to more than 277° C.) and grinding the mixture in a friction ball mill to a fineness of below 5 microns.

4. Emulsifiable Concentrate

An emulsifiable concentrate is obtained from 15 parts by weight of a compound of the general formula (I), 75 parts by weight of cyclohexanone as a solvent and 10 parts by weight of ethoxylated nonylphenol as an emulsifier.

5. Water-Dispersible Granules

Water-dispersible granules are obtained by mixing

75 parts by weight of a compound of the general formula (I),
10 parts by weight of calcium lignosulfonate,
5 parts by weight of sodium laurylsulfate,
3 parts by weight of polyvinyl alcohol and
7 parts by weight of kaolin,
grinding the mixture in a pinned-disk mill, and granulating the powder in a fluidized bed by spray application of water as a granulating liquid.

Water-dispersible granules are also obtained by homogenizing and precomminuting, in a colloid mill,

25 parts by weight of a compound of the general formula (I),
5 parts by weight of sodium 2,2′-dinaphthylmethane-6,6′-disulfonate,
2 parts by weight of sodium oleoylmethyltaurinate,
1 part by weight of polyvinyl alcohol,
17 parts by weight of calcium carbonate and
50 parts by weight of water
then grinding the mixture in a bead mill and atomizing and drying the suspension thus obtained in a spray tower by means of a one-phase nozzle.

### C. Biological Examples

1. Pre-Emergence Herbicidal Action Against Harmful Plants

Seeds of monocotyledonous and dicotyledonous weed plants and crop plants are laid out in sandy loam soil in wood-fiber pots and covered with soil. The compounds of the invention, formulated in the form of wettable powders (WP) or as emulsion concentrates (EC), are then applied to the surface of the covering soil in the form of an aqueous suspension or emulsion at a water application rate equating to 600 to 800 I/ha, with addition of 0.2% wetting agent. After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants). Here, numerous compounds according to the invention showed, at an application rate of 0.32 kg or less per hectare, an activity of at least 80% against a large number of important harmful plants.

At the same time, inventive compounds leave Gramineae crops such as barley, wheat, rye, millet/sorghum, corn or rice virtually undamaged when applied pre-emergence, even at high active ingredient dosages. In addition, some substances are also harmless to dicotyledonous crops such as soya, cotton, oilseed rape, sugar beet or potatoes. Some of the compounds according to the invention exhibit high selectivity and are therefore suitable for controlling unwanted vegetation in agricultural crops by the pre-emergence method. The tables below illustrate, in an exemplary manner, the pre-emergence herbicidal action of the compounds according to the invention, the herbicidal activity being stated in percent.

TABLE 2
Herbicidal pre-emergence action (PE)

| Example | Dosage | Herbicidal action against [%] | | | | | | |
|---|---|---|---|---|---|---|---|---|
| No. | [g/ha] | ECHCG | SETVI | AMARE | VIOTR | ABUTH | ALOMY | POLCO |
| I-09 | 320 | 100 | 90 | 90 | 60 | 70 | 20 | 90 |
| I-03 | 320 | 90 | 40 | 100 | 90 | 80 | 20 | 50 |
| I-07 | 320 | 100 | 90 | 100 | 50 | 80 | 90 | 100 |
| I-05 | 320 | 90 | 90 | 90 | 50 | 50 | 30 | 90 |
| I-06 | 320 | 100 | 90 | 100 | 60 | 20 | 80 | 100 |
| I-16 | 320 | 100 | 80 | 80 | 30 | 80 | 30 | 70 |
| I-04 | 320 | 90 | 70 | 100 | 40 | 90 | 40 | 50 |
| I-01 | 320 | 100 | 90 | 100 | 70 | 80 | 60 | 100 |
| I-08 | 320 | 100 | 90 | 100 | 20 | 0 | 60 | 100 |
| I-02 | 320 | 100 | 90 | 100 | 70 | 30 | 80 | 100 |
| I-13 | 320 | 90 | 100 | 100 | 30 | 30 | 80 | 100 |
| I-36 | 320 | 90 | 90 | 100 | 0 | 90 | 50 | 90 |
| I-20 | 320 | 100 | 100 | 100 | 40 | 50 | 70 | 100 |
| I-10 | 320 | 100 | 90 | 100 | 40 | 30 | 70 | 0 |
| I-12 | 320 | 100 | 90 | 100 | 20 | 20 | 70 | 100 |
| I-18 | 320 | 100 | 70 | 100 | 20 | 40 | 30 | 10 |

TABLE 3
Herbicidal pre-emergence action (PE)

| Exam- | Dosage | Herbicidal action against [%] | | | | |
|---|---|---|---|---|---|---|
| ple No. | [g/ha] | ECHCG | SETVI | AMARE | ABUTH | ALOMY |
| I-42 | 320 | 40 | 20 | 100 | 0 | 30 |
| I-15 | 320 | 30 | 0 | 100 | 90 | 0 |
| I-11 | 320 | 80 | 100 | 100 | 20 | 80 |
| I-21 | 320 | 100 | 80 | 60 | 50 | 40 |
| I-30 | 320 | 100 | 90 | 100 | 20 | 40 |
| I-52 | 320 | 30 | 40 | 80 | 20 | 80 |
| I-29 | 320 | 80 | 70 | 100 | 70 | 80 |
| I-14 | 320 | 100 | 80 | 100 | 50 | 30 |
| I-32 | 320 | 80 | 80 | 100 | 30 | 70 |
| I-31 | 320 | 30 | 50 | 90 | 30 | 20 |
| I-22 | 320 | 90 | 90 | 100 | 20 | 50 |
| I-25 | 320 | 70 | 80 | 100 | 0 | 60 |
| I-47 | 320 | 70 | 50 | 100 | 20 | 60 |
| I-19 | 320 | 50 | 50 | 100 | 70 | 60 |

TABLE 4
Herbicidal pre-emergence action (PE)

| | | |
|---|---|---|
| | | Herbicidal action |
| Example | Dosage | against [%] |

| No. | [g/ha] | ECHCG | AMARE |
|---|---|---|---|
| I-40 | 320 | 10 | 80 |
| I-33 | 320 | 20 | 90 |
| I-28 | 320 | 90 | 100 |
| I-48 | 320 | 10 | 90 |
| I-45 | 320 | 40 | 100 |
| I-37 | 320 | 90 | 100 |
| I-17 | 320 | 90 | 100 |
| I-50 | 320 | 70 | 80 |
| I-34 | 320 | 50 | 100 |
| I-27 | 320 | 60 | 100 |
| I-56 | 320 | 30 | 80 |
| I-59 | 320 | 30 | 80 |
| I-41 | 320 | 90 | 50 |
| I-53 | 320 | 70 | 100 |
| I-24 | 320 | 60 | 90 |
| I-23 | 320 | 90 | 100 |
| I-51 | 320 | 20 | 70 |
| I-35 | 320 | 40 | 100 |
| I-55 | 320 | 40 | 100 |
| I-44 | 320 | 60 | 90 |
| I-46 | 320 | 60 | 100 |
| I-26 | 320 | 70 | 100 |
| I-43 | 320 | 0 | 100 |
| I-54 | 320 | 0 | 80 |

2. Post-Emergence Herbicidal Action Against Harmful Plants

Seeds of monocotyledonous and dicotyledonous weed and crop plants are laid out in sandy loam soil in wood-fiber pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants in the form of an aqueous suspension or emulsion at a water application rate equating to 600 to 800 l/ha, with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants). Here, numerous compounds according to the invention showed, at an application rate of 0.08 kg or less per hectare, an activity of at least 80% against a large number of important harmful plants. At the same time, inventive compounds leave Gramineae crops such as barley, wheat, rye, millet/sorghum, corn or rice virtually undamaged when applied post-emergence, even at high active ingredient dosages. In addition, some substances are also harmless to dicotyledonous crops such as soya, cotton, oilseed rape, sugar beet or potatoes. Some of the compounds according to the invention have high selectivity and are therefore suitable for controlling unwanted vegetation in agricultural crops by the post-emergence method. The tables below illustrate, in an exemplary manner, the post-emergence herbicidal action of the compounds according to the invention, the herbicidal activity being stated in percent.

TABLE 5
Herbicidal post-emergence action (PO)

| Example | Dosage | Herbicidal action against [%] | | | | | | | |
|---|---|---|---|---|---|---|---|---|---|
| No. | [g/ha] | ECHCG | SETVI | AMARE | STEME | VIOTR | ABUTH | PHBPU | POLCO |
| I-18 | 320 | 100 | 90 | 100 | 80 | 90 | 100 | 80 | 80 |
| I-16 | 320 | 90 | 90 | 100 | 80 | 90 | 100 | 80 | 90 |
| I-05 | 320 | 100 | 100 | 100 | 100 | 90 | 100 | 80 | 30 |
| I-09 | 320 | 100 | 90 | 100 | 100 | 90 | 100 | 80 | 70 |
| I-21 | 320 | 90 | 90 | 100 | 90 | 80 | 90 | 80 | 40 |
| I-19 | 320 | 100 | 80 | 100 | 100 | 80 | 90 | 100 | 0 |
| I-35 | 320 | 90 | 100 | 100 | 90 | 60 | 80 | 20 | 80 |
| I-53 | 320 | 90 | 90 | 100 | 100 | 10 | 80 | 0 | 80 |
| I-22 | 320 | 90 | 100 | 100 | 100 | 80 | 100 | 80 | 90 |
| I-04 | 320 | 100 | 90 | 100 | 100 | 50 | 100 | 40 | 80 |
| I-13 | 320 | 90 | 90 | 100 | 100 | 100 | 100 | 80 | 100 |
| I-03 | 320 | 100 | 90 | 100 | 100 | 100 | 100 | 40 | 40 |
| I-40 | 320 | 90 | 90 | 100 | 80 | 60 | 90 | 80 | 40 |
| I-41 | 320 | 100 | 100 | 90 | 100 | 90 | 100 | 70 | 0 |
| I-29 | 320 | 90 | 80 | 100 | 80 | 50 | 80 | 40 | 90 |
| I-07 | 320 | 90 | 100 | 100 | 0 | 60 | 100 | 80 | 80 |
| I-65 | 320 | 80 | 90 | 100 | 80 | 10 | 80 | 0 | 100 |
| I-02 | 320 | 90 | 100 | 100 | 100 | 70 | 90 | 70 | 100 |
| I-34 | 320 | 80 | 100 | 100 | 90 | 0 | 90 | 40 | 80 |
| I-14 | 320 | 90 | 90 | 100 | 100 | 70 | 100 | 80 | 100 |
| I-01 | 320 | 90 | 80 | 100 | 100 | 50 | 100 | 90 | 100 |
| I-55 | 320 | 90 | 90 | 100 | 90 | 10 | 60 | 0 | 90 |
| I-10 | 320 | 90 | 90 | 100 | 100 | 70 | 100 | 80 | 100 |

TABLE 6
Herbicidal post-emergence action (PO)

| | | Herbicidal action | | | |
|---|---|---|---|---|---|
| Example | Dosage | against [%] | | | |
| No. | [g/ha] | SETVI | AMARE | STEME | ABUTH |
| I-33 | 320 | 80 | 100 | 90 | 90 |
| I-49 | 320 | 80 | 100 | 90 | 90 |
| I-38 | 320 | 90 | 100 | 100 | 100 |
| I-31 | 320 | 90 | 100 | 100 | 100 |
| I-12 | 320 | 100 | 100 | 100 | 90 |
| I-57 | 320 | 80 | 100 | 70 | 90 |
| I-52 | 320 | 20 | 90 | 80 | 80 |
| I-56 | 320 | 80 | 90 | 90 | 80 |
| I-08 | 320 | 70 | 100 | 100 | 100 |
| I-20 | 320 | 100 | 100 | 100 | 100 |
| I-39 | 320 | 100 | 90 | 70 | 100 |
| I-24 | 320 | 80 | 100 | 80 | 100 |
| I-15 | 320 | 80 | 100 | 80 | 100 |
| I-45 | 320 | 30 | 90 | 90 | 90 |
| I-06 | 320 | 90 | 100 | 100 | 100 |
| I-47 | 320 | 70 | 90 | 80 | 90 |
| I-30 | 320 | 90 | 100 | 100 | 90 |
| I-37 | 320 | 90 | 100 | 100 | 100 |
| I-11 | 320 | 90 | 100 | 100 | 100 |
| I-36 | 320 | 80 | 100 | 100 | 100 |
| I-64 | 320 | 90 | 90 | 40 | 90 |
| I-44 | 320 | 60 | 100 | 100 | 100 |
| I-48 | 320 | 30 | 90 | 80 | 90 |
| I-25 | 320 | 100 | 100 | 100 | 90 |
| I-59 | 320 | 80 | 90 | 90 | 90 |
| I-42 | 320 | 20 | 90 | 80 | 80 |
| I-26 | 320 | 100 | 100 | 100 | 100 |
| I-23 | 320 | 90 | 100 | 100 | 90 |
| I-28 | 320 | 100 | 100 | 100 | 100 |
| I-27 | 320 | 80 | 100 | 100 | 90 |

TABLE 7
Herbicidal post-emergence action (PO)

| | | Herbicidal action | | | |
|---|---|---|---|---|---|
| Example | Dosage | against [%] | | | |
| No. | [g/ha] | ECHCG | SETVI | AMARE | ABUTH |
| I-69 | 320 | 90 | 80 | 100 | 90 |
| I-60 | 320 | 90 | 80 | 80 | 90 |
| I-43 | 320 | 80 | 90 | 100 | 90 |
| I-50 | 320 | 90 | 40 | 100 | 90 |
| I-17 | 320 | 90 | 100 | 100 | 90 |
| I-54 | 320 | 80 | 80 | 100 | 80 |

TABLE 8
Herbicidal post-emergence action (PO)

| | | Herbicidal action | | | |
|---|---|---|---|---|---|
| Example | Dosage | against [%] | | | |
| No. | [g/ha] | ECHCG | SETVI | AMARE | ABUTH |
| I-69 | 320 | 90 | 80 | 100 | 90 |
| I-60 | 320 | 90 | 80 | 80 | 90 |
| I-43 | 320 | 80 | 90 | 100 | 90 |
| I-50 | 320 | 90 | 40 | 100 | 90 |
| I-17 | 320 | 90 | 100 | 100 | 90 |
| I-54 | 320 | 80 | 80 | 100 | 80 |

TABLE 9
Herbicidal post-emergence action (PO)

| | | |
|---|---|---|
| | | Herbicidal action |
| Example | Dosage | against [%] |

| No. | [g/ha] | AMARE | ABUTH |
|---|---|---|---|
| I-61 | 320 | 90 | 80 |
| I-46 | 320 | 100 | 90 |
| I-58 | 320 | 100 | 90 |
| I-51 | 320 | 90 | 80 |
| I-63 | 320 | 80 | 80 |
| I-32 | 320 | 100 | 90 |
| I-68 | 320 | 80 | 80 |
| I-62 | 320 | 40 | 100 |
| I-71 | 320 | 80 | 40 |

1. A compound of formula (I) 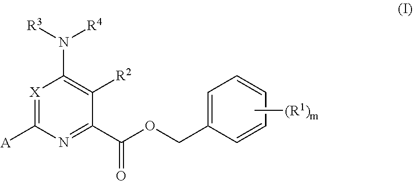
And/or an N-oxide or an agrochemically acceptable salt thereof in which
A represents a radical from the group consisting of A1 to A20, 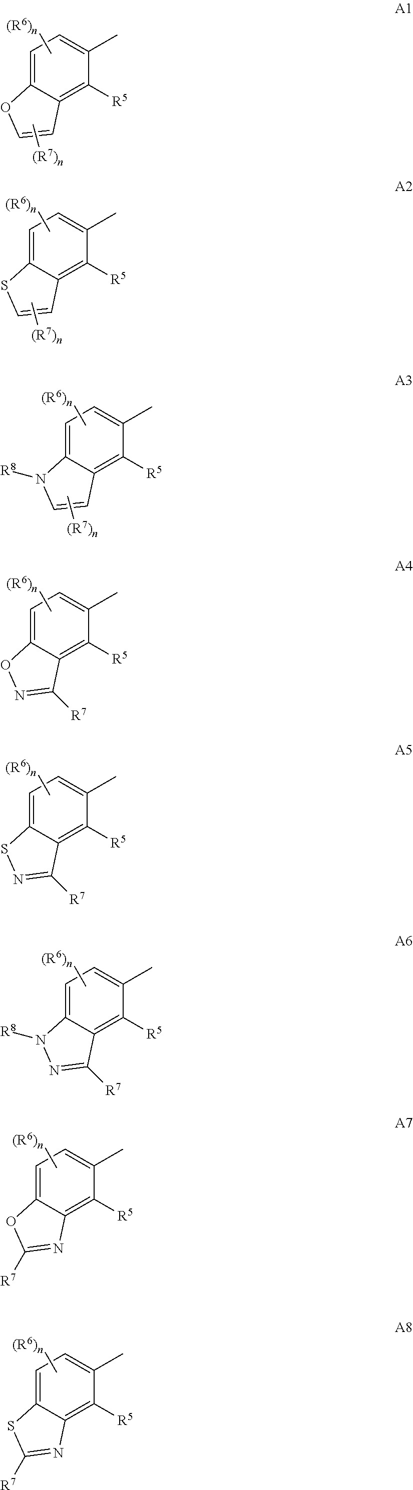 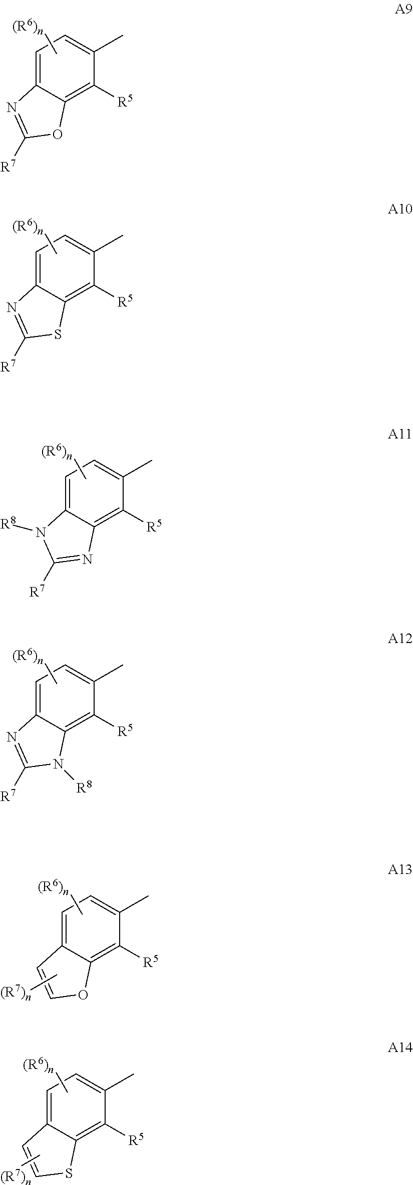 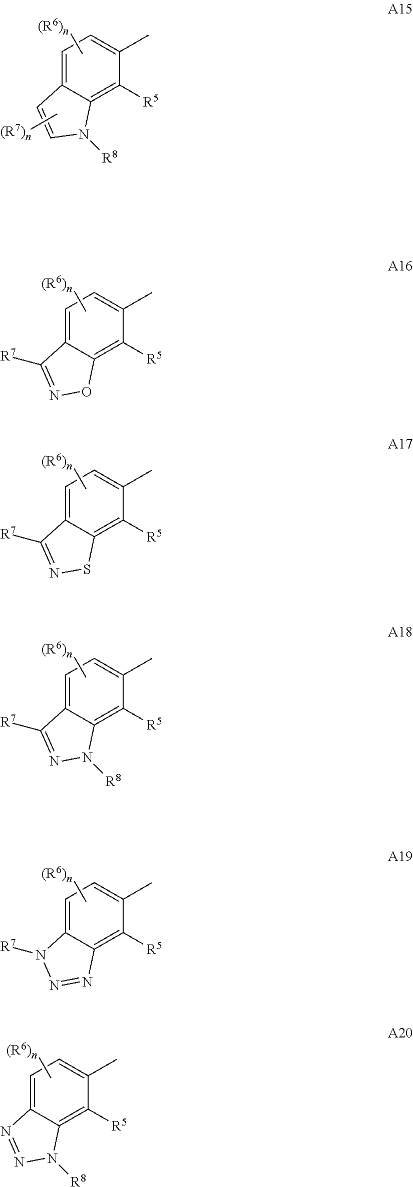
R¹ represents halogen, CN, NO₂, OH, NH₂, (C₁-C₆)-alkyl, (C₁-C₆)-alkoxy, (C₁-C₆)-haloalkyl, (C₁-C₆)-haloalkoxy, (C₂-C₆)-alkenyl, halo-(C₂-C₆)-alkenyl, (C₂-C₆)-alkynyl, halo-(C₃-C₆)-alkynyl, (C₃-C₆)-cycloalkyl, halo-(C₃-C₆)-cycloalkyl, (C₃-C₆)-cycloalkyl-(C₁-C₆)-alkyl, halo-(C₃-C₆)-cycloalkyl-(C₁-C₆)-alkyl, (C₁-C₆)-alkylcarbonyl, (C₁-C₆)-alkylcarboxy, (C₁-C₆)-alkylamine, di(C₁-C₆)-alkylamine, (C₁-C₆)-alkyl-S(O)ₙ or (C₁-C₆)-alkyl-S(O)₂NH,
R² represents chlorine or fluorine,
R³ represents hydrogen,
R⁴ represents hydrogen,
R⁵ represents hydrogen, halogen, OH, NH₂, CN, (C₁-C₃)-alkyl, (C₁-C₃)-alkoxy, C₁-C₃)-alkylamino or cyclopropyl,
R⁶ represents hydrogen, halogen, OH, NH₂, CN, (C₁-C₃)-alkyl, (C₁-C₃)-alkoxy, cyclopropyl or vinyl,
R⁷ represents hydrogen, halogen, (C₁-C₃)-alkyl, (C₁-C₃)-alkoxy, (C₁-C₃)-alkylthio, cyclopropyl, (C₁-C₃)-alkylamino or phenyl,
R⁸ represents hydrogen, (C₁-C₆)-alkyl, (C₁-C₄)-alkylcarbonyl, (C₁-C₆)-alkoxycarbonyl or phenyl,
X represents CH or CF,
m represents 1, 2, 3, 4 or 5 and
n represents 0, 1 or 2.

2. The compound as claimed in claim 1 in which A represents a radical from the group consisting of A1 to A3, A7 to A15 and A17 to A18.

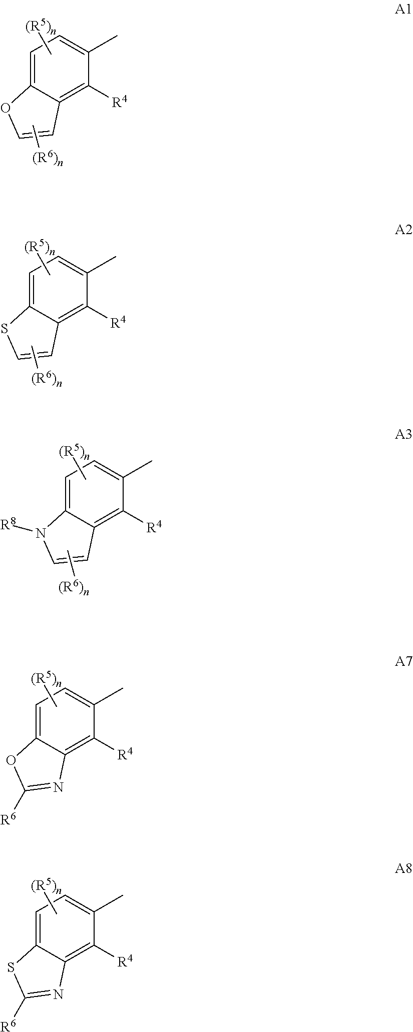

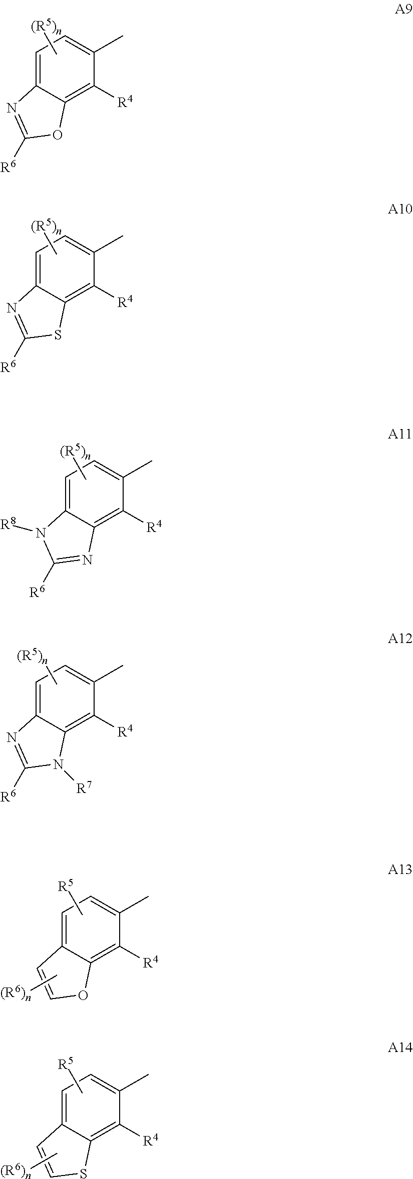

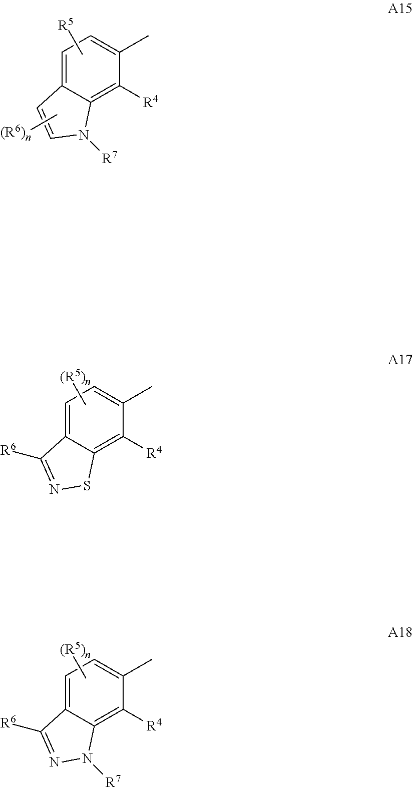

3. The compound as claimed in claim 1 in which R¹ represents halogen, CN, NO₂, OH, NH₂, (C₁-C₆)-alkyl, (C₁-C₆)-alkoxy, (C₁-C₆)-haloalkyl or (C₁-C₆)-haloalkoxy.

4. The compound as claimed in claim 1 in which R² represents chlorine.

5. The compound as claimed in claim 1 in which R³ and R⁴ represent hydrogen.

6. The compound as claimed in claim 1 in which R⁵ represents hydrogen or halogen.

7. The compound as claimed in claim 1 in which R⁶ represents hydrogen or halogen.

8. The compound as claimed in claim 1 in which R⁷ represents hydrogen, halogen or (C₁-C₃)-alkyl.

9. The compound as claimed in claim 1 in which R⁸ represents hydrogen, (C₁-C₄)-alkyl, (C₁-C₄)-alkylcarbonyl or (C₁-C₄)-alkoxycarbonyl.

10. The compound as claimed in claim 1 in which n represents 0 or 1 and m represents 1, 2 or 3.

11. A herbicidal composition comprising a herbicidally active content of at least one compound as claimed in claim 1.

12. The herbicidal composition as claimed in claim 11 in a mixture with one or more formulation auxiliaries.

13. The herbicidal composition as claimed in claim 11, comprising at least one further pesticidally active substance from the group of insecticides, acaricides, herbicides, fungicides, safeners and growth regulators.

14. The herbicidal composition as claimed in claim 11, comprising a safener.

15. The herbicidal composition as claimed in claim 14, comprising a further herbicide.

16. A method of controlling one or more unwanted plants, comprising applying an effective amount of at least one compound as claimed in claim 1 or a herbicidal composition thereof to the plants and/or to a site of unwanted vegetation.

17. A product comprising a compound as claimed in claim 1 or a herbicidal composition thereof for controlling one or more unwanted plants.

18. The product as claimed in claim 17, wherein the compound is used for controlling unwanted plants in one or more crops of one or more useful plants.

19. The product as claimed in claim 18, wherein the useful plants are transgenic useful plants.
